(19) United States
(12) Patent Application Publication
(10) Pub. No.: US 2025/0292114 A1
(43) Pub. Date: Sep. 18, 2025

(54) DISTRIBUTING DIAGNOSTIC PREDICTION MODELS

(71) Applicant: TEMPUS AI, INC., Chicago, IL (US)

(72) Inventors: Raphael Pelossof, New York, NY (US); Talal Ahmed, Highland Park, NJ (US); Mark Carty, Brooklyn, NY (US); Kaveri Nadhamuni, Brooklyn, NY (US)

(21) Appl. No.: 18/607,389

(22) Filed: Mar. 15, 2024

(51) Int. Cl. G06N 5/022; G16H 10/20; G16H 50/20

(52) U.S. Cl. CPC G06N 5/022; G16H 10/20; G16H 50/20

(57) ABSTRACT

Systems and methods for distributing diagnostic prediction models. The system may receive, from an application provider, one or more diagnostic prediction models, each model trained to generate a respective diagnostic prediction based upon genomic data. Adaptation factors may be used to transform a target genomic dataset to conform to a dataset-specific nature of a reference genomic dataset of the model. The system may display via a graphical user interface, one or more representations corresponding to the diagnostic prediction models, and verify a diagnostic prediction model for an application consumer based upon receiving information corresponding to an application consumer genomic dataset. The system may authorize the diagnostic prediction model for distribution to the application consumer, and provide the diagnostic prediction model and the corresponding adaptation factors to the application consumer.

### FIELD OF THE INVENTION

The present disclosure relates to computer-implemented methods and systems for distributing diagnostic prediction models, and more specifically, to distributing diagnostic prediction models trained to make diagnostic predictions without having to share genomic data between a model provider and a model consumer.

### BACKGROUND

The advent of high-throughput gene expression profiling has powered the development of sophisticated molecular models (e.g., diagnostic prediction models). However, successfully applying a prediction model to genomic data other than that on which the model was trained may present multiple issues.

For example, batch-specific effects may exist in genomic datasets based upon the technology, laboratory equipment (e.g., sequencer), and/or library preparation protocols used to generate the genomic data. Correcting batch-specific effects in genomic dataset may require a target dataset (e.g., one on which the model will be prediction applied) and a source dataset which is batch-biased and used as a reference to transform the target dataset when correcting the batch-effects. Therefore, the transfer of prediction models between laboratories or other parties necessitates the transfer of patient-level training data for the prediction model. The need to share and/or access such data can inhibit the transfer of prediction models between laboratories, given the transfer of data may not be possible due to data ownership, the European Union General Data Protection Regulation (GDPR), or similar regulations and/or privacy concerns.

Moreover, genomic datasets used to train a machine learning model may inherently express bias to characteristics of the training data such that when the model is applied to a new source of data that does not express bias to that characteristic, or perhaps expresses bias in a different way, the accuracy or performance of the model may suffer, if not fail altogether, adding another layer of complexity to successfully sharing and implementing prediction models.

Thus, researchers curating models trained on one dataset may find that those models are not transferable to other datasets (e.g., datasets that were not employed for algorithm/model training) due to the model fitting to domain shifts, domain changes target shifts, biases and/or the like of the training dataset, or simply not being configured to process inputs correctly.

At the same time, different entities, such as laboratories, research institutions, health institutions, or pharmaceutical companies may desire to collaborate together while preserving the privacy of their internally curated data. Unable to accomplish their goals without directly sharing data, these entities lack an effective way to work together while maintaining data privacy. In conventional systems, a trusted third party broker is required to facilitate exchanges of molecular models between two entities. This adds costs and complexities, and requires sharing patient-level training data at least for the purpose of assessing whether a given application will function on a new dataset.

Accordingly, there is an unmet need for a technology that enables the distribution of models (e.g., prediction models) in a privacy-preserving manner such that patient data and/or genomic data is not transferred, and the functionality of the models is preserved.

### BRIEF SUMMARY OF THE DISCLOSURE

The disclosed systems and methods address the unmet need for a technology that enables the distribution of models (e.g., prediction models) in a privacy-preserving manner, by eliminating the need for the model provider to share model training data, eliminating the need for the model consumer to share the target genomic data used by the model, eliminating the costs associated with an intermedia data broker, and allowing different entities to collaborate while preserving the privacy of their internally curated data, among other things. Additionally, the disclosed systems and methods may confirm that a shared diagnostic prediction model can generate an expected output when applied to target genomic data of an application consumer before the application consumer is required to purchase the model. Moreover, the disclosed techniques may further eliminate the need for diagnostic prediction models to be re-trained to adapt to new genomic input data through the implementation of a data transformer, saving time, money and resources associated with model training. Furthermore, clinical trial enrollments may be bolstered by applying prognostication models to clinical trial data to find sub-populations responsive to a drug to improve endpoint satisfaction.

In one aspect, a computing system for distributing diagnostic prediction models. The computing system may include one or more memories having stored thereon computer-executable instructions that, when executed by one or more processors, may cause the computing system to: (i) receive from an application provider: (a) one or more diagnostic prediction models, each model trained to generate a respective diagnostic prediction based upon genomic data; and (b) for the each model, adaptation factors used to transform a target genomic dataset to conform to a dataset-specific nature of a reference genomic dataset of the each model; (ii) provide for display via a graphical user interface, one or more representations corresponding to the one or more diagnostic prediction models; (iii) verify a diagnostic prediction model of the one or more diagnostic prediction models for an application consumer based upon receiving information corresponding to an application consumer genomic dataset; (iv) authorize the diagnostic prediction model for distribution to the application consumer; and (v) provide the diagnostic prediction model and the corresponding adaptation factors to the application consumer.

In another aspect, a computer-implemented method for distributing diagnostic prediction models may include: (i) receiving, via one or more processors, from an application provider: (a) one or more diagnostic prediction models, each model trained to generate a respective diagnostic prediction based upon genomic data; and (b) for the each model, adaptation factors used to transform a target genomic dataset to conform to a dataset-specific nature of a reference genomic dataset of the each model; (ii) providing, by the one or more processors for display via a graphical user interface, one or more representations corresponding to the one or more diagnostic prediction models; (iii) verifying, by the one or more processors, a diagnostic prediction model of the one or more diagnostic prediction models for an application consumer based upon receiving information corresponding to an application consumer genomic dataset; (iv) authorizing, by the one or more processors, the diagnostic prediction model for distribution to the application consumer; and (v) providing, by the one or more processors, the diagnostic prediction model and the corresponding adaptation factors to the application consumer.

In yet another aspect, a non-transitory, tangible computer-readable medium storing machine-readable instructions for distributing diagnostic prediction models that, when executed by one or more processors, may cause the one or more processors to: (i) receive from an application provider: (a) one or more diagnostic prediction models, each model trained to generate a respective diagnostic prediction based upon genomic data; and (b) for the each model, adaptation factors used to transform a target genomic dataset to conform to a dataset-specific nature of a reference genomic dataset of the each model; (ii) provide for display via a graphical user interface, one or more representations corresponding to the one or more diagnostic prediction models; (iii) verify a diagnostic prediction model of the one or more diagnostic prediction models for an application consumer based upon receiving information corresponding to an application consumer genomic dataset; (iv) authorize the diagnostic prediction model for distribution to the application consumer; and (v) provide the diagnostic prediction model and the corresponding adaptation factors to the application consumer.

In a further aspect, a computing system for improved trustless diagnostic prediction model sharing includes an electronic network; one or more processors; and one or more memories having stored thereon computer-executable instructions that, when executed by the one or more processors, cause the computing system to: (i) receive from an application provider: one or more diagnostic prediction applications, each including: a diagnostic prediction model which generates a diagnostic prediction based upon a genomic dataset; and a data transformer configured to transform a target genomic dataset to conform a dataset-specific nature of a reference genomic dataset of the each model; and a dataset verifier which identifies at least one cohort of an application consumer genomic dataset for use by a diagnostic prediction application corresponding to the dataset verifier; (ii) receive from an application consumer, a request for a diagnostic prediction application of the one or more diagnostic prediction applications for a corresponding application consumer genomic dataset; (ii) in response to the request for the diagnostic prediction application, transmit to the application consumer, the dataset verifier corresponding to the diagnostic prediction application; (iv) receive from the application consumer, a verified request for the diagnostic prediction application indicating the dataset verifier has identified the at least one cohort of the application consumer genomic dataset for use by the diagnostic prediction application corresponding to the dataset verifier; and (v) in response to the verified request, transmit to the application consumer, the diagnostic prediction application corresponding to the dataset verifier.

In another aspect, a method for automatically verifying a genomic dataset for use with a diagnostic prediction application includes receiving one or more application consumer genomic datasets, and one or more application provider genomic datasets corresponding to a respective one or more diagnostic prediction models; determining, for each application provider genomic dataset of the one or more application provider genomic datasets, a gene-outcome correlation expressed by the each application provider genomic dataset; applying an algorithm to the one or more application consumer genomic datasets to determine, for each application consumer genomic dataset of the one or more application consumer genomic datasets, whether the gene-outcome correlation expressed by the each application provider genomic dataset is substantially similar to the gene-outcome correlation expressed by the one or more application consumer genomic datasets, wherein the gene-outcome correlation expressed by at least one of the one or more application consumer genomic datasets is substantially similar to the gene-outcome correlation expressed by at least one of the one or more application consumer genomic datasets, generating a gene-outcome validation dataset indicating the substantial similarities; and wherein the gene-outcome correlation expressed by at least one of the one or more application consumer genomic datasets is not substantially similar to the gene-outcome correlation expressed by at least one of the one or more application provider genomic datasets, applying the algorithm to the at least one of the one or more application consumer genomic datasets which are not substantially similar and the one or more application provider genomic datasets to determine pairs of genes which have at least one covariance between the at least one of the one or more application consumer genomic datasets which are not substantially similar and a respective application provider genomic dataset; and generating a gene-gene validation dataset indicating the covariance between the at least one of the one or more application consumer genomic datasets which are not substantially similar to a respective application provider genomic dataset.

In another aspect, a method for rescuing failed drugs includes receiving a diagnostic prediction model trained to prognosticate a genomic condition based upon a genomic dataset; analyzing, by the diagnostic prediction model, an historical genomic dataset indicative of a failed trial to generate a prognostication of the genomic condition; and outputting a report indicating the prognostication of the genomic condition.

In yet another aspect, a computer-implemented method for providing a plugin to an electronic health record system via a plug-in architecture of the electronic health record system includes displaying, via a graphical user interface of an electronic health records client, a representation of one or more diagnostic prediction models, each trained to generate a respective diagnostic prediction based upon genomic data of an electronic health record; receiving, via the graphical user interface, a selection of at least one diagnostic prediction model of the one or more diagnostic prediction models; generating, at the electronic health records client, an electronic request including (i) an indication of the at least one diagnostic prediction model selected and (ii) one or more electronic health records of a patient including at least some genomic data; transmitting, via an electronic network, the electronic request to a remote computing device; receiving, from the remote computing device, an electronic result including one or more diagnostic predictions generated by the at least one diagnostic prediction models; and displaying, via the graphical user interface, the electronic result.

In accordance with the above, and with the disclosure herein, the present disclosure includes improvements in computer functionality or in improvements to other technologies at least because the claims recite, e.g., distributing diagnostic prediction models without the need to share model training data by the application provider, or share the target genomic dataset by the application consumer which will be used as an input the model. The application provider may provide adaptation factors which may be used to transform the target genomic dataset to conform a dataset-specific nature of a reference/training genomic dataset of the model. The dataset-specific may include batch effects; population, domain, and/or covariate changes or shifts; biases, and/or other dataset-specific phenomena. In addition, the disclosed systems and methods for distributing models in a privacy-preserving manner can be applied to many multiples (e.g., hundreds or thousands) of different models, each having different predictive and/or other functions, different biases, batch effects, and/or population/domain changes in the training data, being applied to different consumer genomic datasets also having their own biases, batch effects and/or population/domain changes, allowing verification of a customer genomic dataset before the model is purchased, etc. That is, the model distribution systems and methods remove the burden of sharing private data, and allow collaboration between different parties, advance the field of genomic research conducted via the models, when distributing models in a new more efficient and/or streamlined manner when compared to existing model sharing approaches. For example, a diagnostic predictive model which predicts the occurrence of cancer may be trained by a research institution on a private genomic dataset generated by a first set of sequencing equipment during dozens of trials. The medical provider may wish to apply the model to genomic datasets of tens, hundreds or even thousands of patients in their healthcare system who may be at risk of cancer to provide preventative health services, however the patient genomic datasets may be generated by numerous types of sequencing equipment. Privacy and/or research laws, regulation and/or agreements may prohibit each party from sharing their genomic data, stunting benefits which may flow to the research institution (e.g., receiving funds for use of the model on such a large populous of people, which may fund further research), and preventing the patients of the healthcare system from receiving potentially life-saving predictions which may result in preventative healthcare. That is, the present disclosure describes improvements in the functioning of a computer itself or “any other technology or technical field” because in sharing the model and adaptation factors without sharing genomic data of the provider or consumer of the model, and entire new electronic model sharing ecosystem is created. Further improvements are also described when using a computing environment to verify the genomic data for suitable use and/or compatibility with the model, before requiring purchase of the model and likewise without the necessity of sharing genomic data on the part of the application provider and/or consumer. Moreover, the technical field of model-based genomic research is improved by providing the ability for different parties to share models to advance the research, predictive capabilities of the models, etc., for parties which may otherwise not have had access to the models due to data privacy and/or other restrictions or concerns. These are improvements over the prior art at least because system, digital marketplaces and/or platforms, which would otherwise be required to receive, store and share model consumer and/or provider genomic datasets to enable model interoperability between a voluminous number of parties, which would require increased memory storage and/or execution compute cycles by processing devices, no longer need to store and process genomic data to distribute models.

The present disclosure includes specific features other than what is well-understood, routine, conventional activity in the field, and/or otherwise adds unconventional steps that confine the disclosure to a particular useful application, e.g., systems and methods for distributing diagnostic prediction models.

Additional, alternate and/or fewer actions, steps, features and/or functionality may be included in an aspect and/or embodiments, including those described elsewhere herein.

### BRIEF DESCRIPTION OF THE DRAWINGS

The figures described below depict various aspects of the applications, methods, and systems disclosed herein. It should be understood that each figure depicts one embodiment of a particular aspect of the disclosed applications, systems and methods, and that each of the figures is intended to accord with a possible embodiment thereof. Furthermore, wherever possible, the following description refers to the reference numerals included in the following figures, in which features depicted in multiple figures are designated with consistent reference numerals.

FIG. 1 depicts a block diagram of an exemplary computer system in which methods and systems for distributing diagnostic prediction models are implemented, according to some aspects.

FIG. 2A depicts a block diagram of an exemplary system for trustless sharing of diagnostic prediction models, according to some aspects.

FIG. 2B depicts an exemplary GUI for distributing diagnostic prediction models, according to some aspects.

FIG. 2C depicts an exemplary GUI for training diagnostic prediction models, according to some aspects.

FIG. 2D depicts a block diagram of an exemplary system for distributing verifiable diagnostic prediction models, according to some aspects.

FIG. 3A depicts an exemplary graph of molecular reproducibility results of gene-association correlations for responses to carboplatin in Endometrioid cancers of two genomic datasets, according to some aspects.

FIG. 3B depicts an exemplary graph of molecular reproducibility results of gene-association correlations for responses to carboplatin in Endometrioid cancers of two genomic datasets, according to some aspects.

FIG. 4 depicts an exemplary table of gene-gene correlation results for training and validation genomic datasets of a PAM50 breast cancer subtyping model, according to some aspects.

FIG. 5A illustrates an exemplary graph of results for applying a prognosticator to a training genomic dataset and drug trial genomic dataset respectively, according to some aspects.

FIG. 5B illustrates an exemplary graph of results for applying a prognosticator to a training genomic dataset and drug trial genomic dataset respectively, according to some aspects.

FIG. 6 depicts a flow diagram of an exemplary computer-implemented method for distributing diagnostic prediction models, according to some aspects.

FIG. 7 depicts a flow diagram of an exemplary computer-implemented method for automatically verifying a genomic dataset for use with a diagnostic prediction application, according to some aspects.

FIG. 8 depicts a flow diagram of an exemplary computer-implemented method for rescuing failed drugs, according to some aspects.

FIG. 9 depicts a flow diagram of an exemplary computer-implemented method for providing a plugin to an electronic health record system via a plug-in architecture of the electronic health record system, according to some aspects.

FIG. 10A depicts an exemplary model exchange environment, according to some aspects.

FIG. 10B depicts an exemplary model exchange environment including verification, according to some aspects.

FIG. 10C depicts an exemplary model exchange environment, scaled to many consumer and producer participants, according to some aspects.

FIG. 11 depicts an exemplary cosine similarity matrix, according to some aspects.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The systems and methods described herein address the aforementioned unmet needs and deficiencies by distributing diagnostic prediction models (DPMs) trained on an application provider's genomic dataset, to an application consumer for application of the DPM to a target consumer genomic dataset, without requiring the sharing of the application consumer and/or provider genomic datasets for the DPM to operate successfully. The systems and methods described may distribute adaptation factors, data transformers, coefficients, and/or other electronic mechanism(s) for purposes such as identifying the nature of batch effects or population/domain changes, biases, domain shifts, domain changes, covariate shifts, and/or other dataset specific phenomenon in a source genomic dataset (e.g., the training data of the DPM or using an existing algorithm on a new domain) and/or transforming a target genomic dataset (e.g., application consumer genomic dataset) to conform to the a dataset-specific nature of the source genomic dataset (e.g., batch effects, population or domain changes, etc.) without requiring sharing of the source and/or target genomic data. Further, the present techniques may distribute a dataset verifier to verify at least a cohort of an application consumer's genomic dataset for use by the DPM of an application provider before the DPM is sold and/or distributed to the application provider, providing verifiability that that the DPM can operate on the cohort to a minimum standard.

Accordingly, the present techniques are directed to computer-implemented methods and systems for distributing diagnostic prediction models, and more specifically, for distributing diagnostic prediction models trained to make diagnostic predictions without having to share genomic data between a model provider and a model consumer. The present techniques remove the requirement to share patient-level training data to assess applications on new domain(s), and provide measures of confidence that downloaded applications/models will maintain high levels of accuracy when applied to molecular data from the new domain(s).

### Example Computer Environment

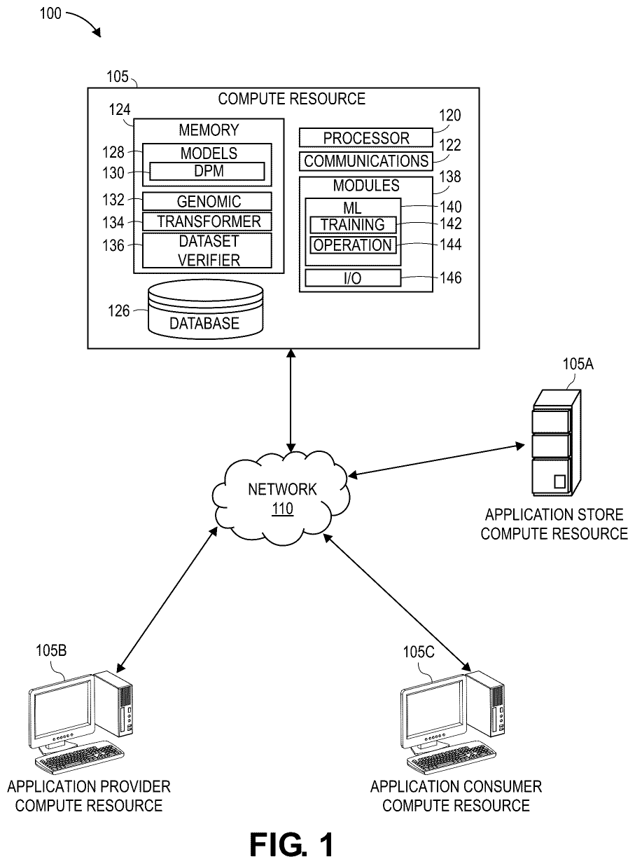

FIG. 1 depicts an example computing environment 100 for distributing diagnostic prediction models, according to some aspects. Although FIG. 1 depicts certain entities, components, equipment, and devices, it should be appreciated that additional or alternate entities, components, equipment, and devices are envisioned.

As illustrated in FIG. 1, the computing environment 100 may include one or more compute resources 105 that may perform operations such as verifying, distributing, and/or receiving genomic datasets and/or models (e.g., the DPM).

The compute resources 105 may be part of a cloud network or may otherwise communicate with other hardware or software components within one or more cloud computing environments to send, retrieve, or otherwise analyze data or information described herein. For example, in certain aspects of the present techniques, the computing environment 100 may comprise an on-premise computing environment, a multi-cloud computing environment, a public cloud computing environment, a private cloud computing environment, and/or a hybrid cloud computing environment. For example, an entity (e.g., a business) may host one or more services in a public cloud computing environment (e.g., Amazon Web Services (AWS), Google Cloud, IBM Cloud, Microsoft Azure, etc.). The public cloud computing environment may be an off-premise cloud (e.g., not physically hosted at a location owned by the business). Alternatively, or in addition, aspects of the public cloud may be hosted on-premise at a location owned by the business. The public cloud may be partitioned using visualization and multitenancy techniques and may include one or more software-as-a-service (SaaS), infrastructure-as-a-service (IaaS) and/or platform-as-a-service (PaaS) services. One or more functionalities of the compute resource 105 (e.g., model training, data transforming, model verification, diagnostic predictions via model) may be implemented and/or offered for sale via SaaS, IaaS and/or PaaS modalities. The electronic network 110 may include one or more networks, including a local area network (LAN), a wide area network (WAN), the Internet, a mesh network, and/or another suitable electronic network. For example, the network 110 may include a wireless cellular service (e.g., a 4G network, a 5G network, a 6G network, etc.). Generally, the network 110 enables bidirectional communication between the compute resources 105, and optionally, other networked components (not depicted) such as one or more mobile computing devices, one or more network routers and/or one or more network switches.

The computing environment 100 may include a network 110. The network 110 may comprise a cellular base station, such as cell tower(s), communicating to the one or more components of the computing environment 100 via wired/wireless communications based upon any one or more of various mobile phone reference standards, including NMT, GSM, CDMA, UMTS, LTE, 5G, 6G, or the like. Additionally, or alternatively, the network 110 may include other such wireless connection points communicating to the components of the computing environment 100 via wireless communications based upon any one or more of various wireless references, including IEEE 802.11a/ac/ax/b/g/n (Wi-Fi), Bluetooth, etc.

A communications component 122 may allow the compute resource 105 to communicate over the network 110 via any suitable wired and/or wireless connection, e.g., using any suitable network interface controller(s) of the communications component 122. The communications component 122 may include one or more transceivers (e.g., WWAN, WLAN, and/or WPAN transceivers) functioning in accordance with IEEE reference standards, 3GPP reference standards, and/or other reference standards that may be used in receipt and transmission of data via external/network ports of the compute resource 105 connected to the network 110.

The compute resource 105 may include one or more processors 120. The processors 120 may include one or more suitable processors (e.g., central processing units (CPUs) and/or graphics processing units (GPUs)). The processors 120 may be communicatively coupled to a memory 124 via a computer bus (not depicted) responsible for transmitting electronic data, data packets, or otherwise electronic signals to and from the processors 120 and the memory 124 in order to execute, implement or perform the machine-readable instructions, methods, processes, elements, or limitations, as illustrated, depicted, or described for the various flowcharts, illustrations, diagrams, figures, and/or other disclosure herein. The processors 120 may interface with the memory 124 via the computer bus to execute an operating system (OS) and/or computing instructions contained therein, and/or to access other services/aspects. For example, the processors 120 may interface with the memory 124 via the computer bus to create, read, update, delete, or otherwise access or interact with the data stored in the memory 124, database 126, and/or another source of data.

The memory 124 may include one or more forms of volatile and/or nonvolatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 124 may store an operating system (e.g., Microsoft Windows, Linux, UNIX, etc.) capable of facilitating the functionalities, apps, methods, or other software as described herein. The memory 124 may store one or more sets of non-transitory, computer-executable instructions that, when executed, cause the compute resource 105 to perform certain functions.

In general, a computer program or computer based product, application, or code (e.g., the model(s), such as machine learning (ML) models, or other computing instructions described herein) may be stored on a computer usable storage medium, or tangible, non-transitory computer-readable medium (e.g., reference random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 120 (e.g., working in connection with the respective operating system in the memory 124) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective C, Java, Scala, ActionScript, JavaScript, HTML, CSS, XML, etc.).

The memory 124 may include, or be communicatively coupled to (e.g., via network 110), one or more electronic databases 126. The databases 126 may include a relational database, such as Oracle, DB2, MySQL, a NoSQL database, such as MongoDB, or another electronic database. The databases 126 may store, for example, genomic datasets, models, or other suitable data.

The memory 124 and/or databases 126 may include one or more genomic datasets 135, e.g., genomic datasets of a diagnostic application/model consumer, of a diagnostic application/model provider, public datasets, a laboratory-specific datasets, historical datasets, genomic datasets of electronic health records (EHRs), and/or other genomic datasets.

The memory 124 and/or the databases 126 may store one or more models, routines, algorithms, or other elements (e.g., one or more models 128). The models 128 may be referred to as receiving an input, producing or storing an output, or executing as instructions on the processor 120. Further, the models 128 may be stored in the memory 124 as computer-executable instructions, which instructions the processor 120 may access and execute. Further, the processor 120 should be understood to retrieve/access from the memory 124 any data necessary to perform the executed instructions (e.g., data required as an input to the routine or model), and to store in the memory 124 the intermediate results and/or output of any executed instructions.

In at least some aspects, the models 128 may include one or more machine learning (ML) models, as further described below. The models 128 may include a DPM 130 trained to generate a diagnostic prediction based upon receiving genomic data. In at least some aspects, the DPM 130 may be a diagnosticator model (e.g., a model that classifies and/or identifies a condition), a prognosticator model (e.g., to predict a future outcome), a subtyping model (e.g., a model that performs molecular subtyping, identifies a disease by form or stage, and/or that otherwise segments a population), a predictive model (e.g., make future predictions of an outcome), or any other suitable type of model to generate a prediction and the like based upon genomic data. In at least some aspects, the DPM 130 may be a machine-learning model trained using historical genomic data to make diagnostic predictions, such as a prediction of a risk of cancer occurrence. In at least some aspects, the diagnostic prediction may include a validation score or other metric indicating a validity of each diagnostic prediction based upon compatibility of the genomic data of the diagnostic prediction with the DPM 130. For example, the DPM 130 may generate a risk of developing breast cancer based upon a genomic dataset of a patient.

The memory 124 and/or databases 126 may store one or more genomic datasets 132. The genomic datasets 132 may include labels, genomic expression data, model training data (e.g., testing, validation, feedback, and/or other training data), adaptation factors of the genomic datasets 132, etc. The genomic datasets 132 may be used to create, train, operate, and/or fine-tune the models 128 (e.g., DPM 130), generate a prediction, generate adaptation factors, and/or other suitable functions. The genomic datasets 132 may be and/or include publicly available genomic data, genomic data generated by sequencing equipment of a laboratory, etc.

The memory 124 and/or databases 126 may include one or more data transformers 134. The data transformers 134 may be or include an algorithm, model, application, program, and the like, to transform a target genomic dataset to conform to a dataset-specific nature of a reference genomic dataset, such data specific phenomena being referred to herein as adaptation factors. For example, the data transformers 134 may use adaptation factors of the training genomic dataset to transform an application consumer genomic dataset batch effects; population, domain, and/or covariate changes or shifts; biases, and/or other dataset-specific phenomena, to conform to one or more batch effects; population, domain, and/or covariate changes or shifts; biases, and/or other dataset-specific phenomena of a training genomic dataset used to train the DPM 130. Transforming application consumer genomic datasets using data transformers 134 can allow the DPM 130 to be applied to the application consumer genomic dataset to make a prediction, without having to retrain the DPM 130. In at least some aspects, the data transformers 134 associated with the DPM 130 may also include the adaptation factors of the DPM 130.

In addition to, or alternative to, transforming a target genomic dataset to conform to batch effects or population/domain changes, the DPM 130 may be applied to a target genomic dataset (e.g., of a DPM consumer) which has not been transformed using adaptation factors and/or the data transformers 134. In such an example, the DPM 130 may not be able to provide a diagnostic prediction or may provide a diagnostic prediction which may be accurate, partially accurate, or inaccurate, (e.g., as indicated by an associated metric such as a confidence score) when applying the DPM 130 to the non-transformed target genomic dataset. However, transforming the target genomic dataset may enable the DPM 130 to provide a diagnostic prediction, and/or improve the accuracy/suitability of the prediction, when applied to the transformed target genomic dataset.

In at least some embodiments, the DPM 130 and one or more of the adaptation factors of the genomic dataset 132 corresponding to the DPM 130 (e.g., the DPM genomic training data) and/or the data transformers 134 may be referred to, and/or include, a diagnostic prediction application (DPA). In at least some aspects, an application consumer may desire to purchase the DPA from the application provider to run the DPA on their target genomic dataset. The application consumer may want to transform a target genomic dataset to conform to the dataset-specific nature of the training genomic dataset of the DPA before applying the DPA to their target genomic dataset, e.g., to improve the diagnostic prediction the DPA provides, as just described. However, the application consumer may not want to share their target genomic dataset with the application provider due to privacy or other aforementioned concerns, and would rather use their own means to transform their target genomic dataset. As the DPA includes the adaptation factors and/or data transformers 134, the application consumer has all the information necessary data and/or tools to determine the batch effects or population/domain changes of the genomic training data of the DPA they are purchasing, and use this information to transform their own, target genomic dataset to conform to the dataset-specific nature of the DPM 130 genomic training data.

The memory 124 and/or databases 126 may include a dataset verifier 136, which may be associated with one or more DPMs 130. The dataset verifier 136 may be or include an algorithm, model, application and/or computer program. The dataset verifier 136 may be applied to a genomic dataset 132 to identify one or more cohorts of the genomic dataset 132 most suitable and/or compatible for use by the associated DPM 130, e.g., suitable and/or compatible for the DPM 130 to make a prediction on the verified cohort(s) of the genomic data. In at least some aspects, the dataset verifier 136 may be applied to one or more genomic datasets 132 (e.g., application consumer genomic datasets) to determine one or more DPMs 130 which may be compatible with the genomic datasets. In one example, an application consumer may have a genomic dataset 132 which they would like to use as an input for one or more DPMs 130 available for purchase from the application provider. Before purchasing the DPM 130, the application consumer may want to confirm that the DPM 130 is compatible with at least a cohort of their genomic dataset. The application provider may not want to provide the DPM 130 to the application consumer to make such a confirmation without the application consumer purchasing the DPM 130. In such an example, the application provider may provide to the consumer one or more dataset verifiers 136 to evaluate whether one or more associated DPMs 130 associated with the dataset verifier 136 may be compatible with at least a cohort of the application consumer's genomic dataset. In doing so, the dataset verifier 136 may provide the application consumer a measure of confidence that one or more compatible DPMs 130 will successfully operate on at least a cohort of their genomic dataset, making the purchase worthwhile.

In at least some aspects, the dataset verifier 136 may confirm a target genomic dataset is suitable and/or compatible for use with an associated DPM 130 based upon a gene and associated outcome (gene-outcome) correlation or a gene-gene correlation between a source genomic dataset (e.g., genomic dataset used to train the DPM 130) and a target genomic dataset (e.g., genomic dataset of a consumer who desires to apply the DPM 130 to their genomic dataset). The dataset verifier 136 may determine a gene-outcome correlation when there is at least one relevant label in common between the source genomic data, and data of the target genomic dataset being verified for application to the DPM 130. In at least some aspects, the gene-outcome is based upon risk genes and protective genes of the genomic datasets. If a gene-outcome correlation does exist between the source genetic dataset and at least a cohort of the target genomic dataset, the dataset verifier 136 may verify the target genomic dataset (or at least the cohorts of the gene-outcome correlation) for use by the associated DPM 130.

The dataset verifier 136 may verify a target genomic dataset as suitable and/or compatible for use by an associated DPM 130 when determining a gene-gene correlation of pairs of genes which have at least one covariance between the target genomic data (e.g., application consumer genomic dataset) and at least a cohort of the source genomic data (e.g., dataset used to train the DPM 130) for the associated DPM 130. This gene-gene correlation may include generating a cosine similarity between the target and source genomic datasets. In at least some aspects, the dataset verifier 136 may only evaluate a gene-gene correlation between the source and target genomic datasets when there is no relevant label in common between the target and source genomic dataset, e.g., when there is no gene-outcome correlation between the source and target genomic datasets.

The memory 124 may store a plurality of computing modules 138, implemented as respective sets of computer-executable instructions (e.g., one or more source code libraries, trained ML models such as neural networks, convolutional neural networks, etc.) as described herein. For example, although depicted in FIG. 1 as part of memory 124, the transformers 134 and/or the dataset verifier 136 may be considered computing modules 138.

In one aspect, the computing modules 138 may include an ML module 140. The ML module 140 may include ML training module (MLTM) 142 and/or ML operation module (MLOM) 144. In some embodiments, at least one of the models 128 may be applied by the ML module 140, which may include, but are not limited to linear or logistic regression algorithms, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of ML, such as supervised learning, unsupervised learning, and reinforcement learning.

In one aspect, the ML based algorithms may be included as a library or package executed on compute resource(s) 105. For example, libraries may include the TensorFlow based library, the Pytorch library, and/or the scikit learn Python library.

In one embodiment, the ML module 140 employs supervised learning, which involves identifying patterns in existing data to make predictions about subsequently received data. Specifically, the ML module provides model “training” (e.g., via MLTM 142) using input-output pairs of training data to learn a mapping from input to output, e.g., maps clinical-molecular data to one or more predictions which may include risk assessment, subtyping, diagnostic, prognostic, and/or predictive scores. The exemplary inputs and exemplary outputs of the training data may include any of the data inputs or ML outputs described above. In the exemplary embodiments, a processing element may be trained by providing it with a large sample of data with known characteristics or features. Once trained, the ML model can accurately predict the output when provided new, unknown inputs,

In another embodiment, the ML module 140 may employ unsupervised learning, which involves finding meaningful relationships in unorganized data. Unlike supervised learning, unsupervised learning does not involve user-initiated training based upon exemplary inputs with associated outputs. Rather, in unsupervised learning, the ML module 140 may organize unlabeled data according to a relationship determined by at least one ML method/algorithm employed by the ML module 140. Unorganized data may include any combination of data inputs and/or ML outputs as described above.

In yet another embodiment, the ML module 140 may employ reinforcement learning, which involves optimizing outputs based upon feedback from a reward signal. Specifically, the ML module 140 may receive a user-defined reward signal definition, receive a data input, utilize a decision making model to generate the ML output based upon the data input, receive a reward signal based upon the reward signal definition and the ML output, and alter the decision making model so as to receive a stronger reward signal for subsequently generated ML outputs. Other types of ML may also be employed, including deep or combined learning techniques.

The MLTM 142 may receive labeled data at an input layer of a model having a networked layer architecture (e.g., an artificial neural network, a convolutional neural network, etc.) for training the one or more ML models. The received data may be propagated through one or more connected layers of the ML model to establish weights of one or more nodes, or neurons, of the respective layers. Initially, the weights may be initialized to random values, and one or more suitable activation functions may be chosen for the training process. The present techniques may include training a respective output layer of the one or more ML models. The output layer may be trained to output a diagnostic prediction, for example, based upon receiving genomic data as an input.

The MLOM 144 may comprise a set of computer-executable instructions implementing ML loading, configuration, initialization and/or operation functionality. The MLOM 144 may include instructions for storing trained ML models (e.g., as ML models in memory 124). As described, once trained, the one or more trained ML models may be operated in inference mode, whereupon when provided with de novo input that the model has not previously been provided, the model may output one or more predictions, classifications, etc., as described herein.

In operation, ML model training module 142 may access memory 124, databases 126, and/or any other data source for training data suitable to generate one or more ML models, such as models 128. The training data may be sample data with assigned relevant and comprehensive labels (classes or tags) used to fit the parameters (weights) of an ML model with the goal of training it by example. In one aspect, once an appropriate ML model is trained and validated to provide accurate predictions and/or responses, the trained ML model may be loaded into MLOM 144 at runtime to process input data and generate output data.

While various embodiments, examples, and/or aspects disclosed herein may include training and generating one or more ML models for the compute resource 105 to load at runtime, one or more appropriately trained ML models may already exist (e.g., models 128 in memory 124) such that the compute resource 105 may load an existing trained ML model at runtime. It is further contemplated that the compute resource 105 may retrain, finetune, update and/or otherwise alter an existing ML model before and/or after loading the model at runtime.

In one aspect, the computing modules 138 may include an input/output (I/O) module 146, comprising a set of computer executable instructions implementing communication functions. The I/O module 146 may include a communication component configured to communicate (e.g., send and receive) data via one or more external/network port(s) to one or more networks or local terminals, such as the network 110 described herein. In one aspect, the compute resources 105 may include a client-server platform technology such as ASP.NET, Java J2EE, Ruby on Rails, Node.js, a web service or online API, responsive for receiving and responding to electronic requests.

The I/O module 146 may further include or implement a user interface configured to present information to an administrator, operator or other user, and/or receive inputs from the user, such as via a touchscreen display. The I/O module 146 may facilitate I/O components (e.g., ports, capacitive or resistive touch sensitive input panels, keys, buttons, lights, LEDs), which may be directly accessible via, or attached to, the compute resource 105 and/or may be indirectly accessible via, or attached to, another device. According to one aspect, the user may access the compute resource 105 via a user interface to review information, make changes, input training data, initiate training via the MLTM 142, and/or perform other functions (e.g., operation of one or more trained models via the MLOM 144).

The computing environment 100 may include multiple compute resources such as compute resource 105, for example compute resources operated and/or associated with different entities such as an application store compute resource 105A, an application provider compute resource 105B, and/or an application consumer compute resource 105C. In at least some embodiments, one or more of the compute resources 105A, 105B, 105C may be affiliated with a biotechnology entity, an educational entity, a collaborator of a provider of the diagnostic prediction models, a pharmaceutical entity, and/or the like. For example, the application store compute resource 105A may receive one or more models 128 and/or applications such as DPMs 130 and/or DPAs respectively, adaptation factors associated with reference genomic data (e.g., genomic training data) of the models 128, data transformers 134 and/or dataset verifiers associated with the models 128, among other things. The application store compute resource 105A may offer one or more of the models 128 for sale. One or more application consumers may subscribe, purchase, and/or otherwise obtain models 128 e.g., via application consumer compute resource 105C. The application store compute resource 105A and/or application consumer compute resource 105C may be able to verify consumer genomic data (e.g., via dataset verifier 136) as suitable for use with one or more associated models 128. These compute resources 105A, 105C may transfer the verified models 128 from the application store compute resource 105A to the application consumer compute resource 105C without having to transfer the underlying source/training genomic data of the model 128 from the application provider to the application consumer, and/or without having to transfer the application consumer/target genomic data from the application consumer to the application provider for the model 128 to operate on the application consumer/target genomic data. Accordingly, one or more models 128 may be shared between two entities that wish to transfer models 128 without having to share (trustless sharing) patient-level data to assess genomic datasets used by the model 128, while having a measure of confidence that a model 128 may output a valid prediction on a genomic dataset other than that which it has been trained to operate upon.

Although the computing environment 100 is shown to include one each of compute resources 105, 105A, 105B, 105C and one network 110 it should be understood that different numbers of compute resources 105, 105A, 105B, 105C, and/or networks may be utilized. In one example, the computing environment 100 may include hundreds of compute resources 105, 105A, 105B all of which may be interconnected via the network 110.

The computing environment 100 may include additional, fewer, and/or alternate components, and may be configured to perform additional, fewer, or alternate actions, including components/actions described herein. For example, although the compute resource 105 is shown in FIG. 1 as including one instance of various components such as processor 120, memory 124 and database 126, various aspects include the computing environment 100 and/or compute resource 105 implementing any suitable number of any of the components shown in FIG. 1 and/or omitting any suitable ones of the components shown in FIG. 1. For instance, information described as being stored at memory 124 may be stored at databases 126, and therefore memory 124 may be omitted. Moreover, various aspects include the computing environment 100 including any suitable additional component(s) not shown in FIG. 1, such as but not limited to the exemplary components described above. Furthermore, it should be appreciated that additional and/or alternative connections between components shown in FIG. 1 may be implemented. As just one example, compute resource 105 may be connected to database 126 via network 110 rather than appearing to be connected via a direct connection in FIG. 1.

### Trustless Sharing of Diagnostic Prediction Models

The present techniques may support multiple configurations for training and using models. For example, in one aspect, a consumer may edit (e.g., via the application consumer compute resource 105C) their own data and the original model can be run on the changed data at the consumer's computing environment. In another aspect, the store (e.g., via the application store compute resource 105A) trains the model with an altered source data and the changed model is run on the consumer's unchanged data. The application store compute resource 105A may include a database that stores all of the existing trained basis for the expected domain, then when a new domain comes in from a potential client, check to see if there already exists a model trained for that basis and deliver that one without retraining the model. Basis is the main axis along which the training data is distributed. Classic basis would be PCA orthonormal basis. Other decomposition algorithms and embedding algorithms can create non-linear bases as well.

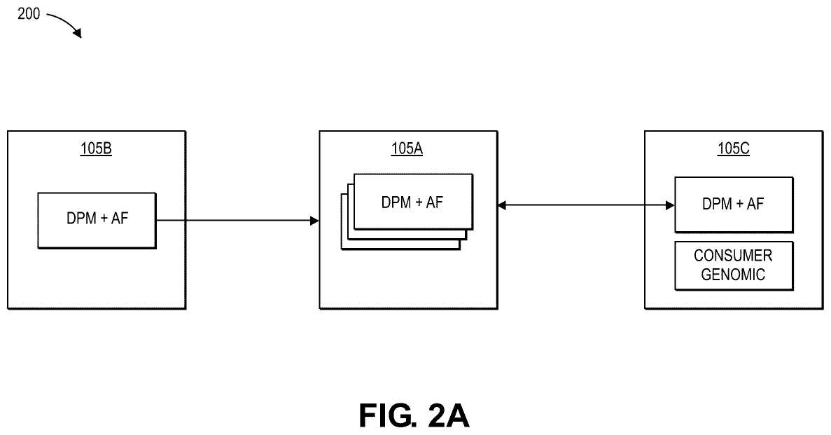

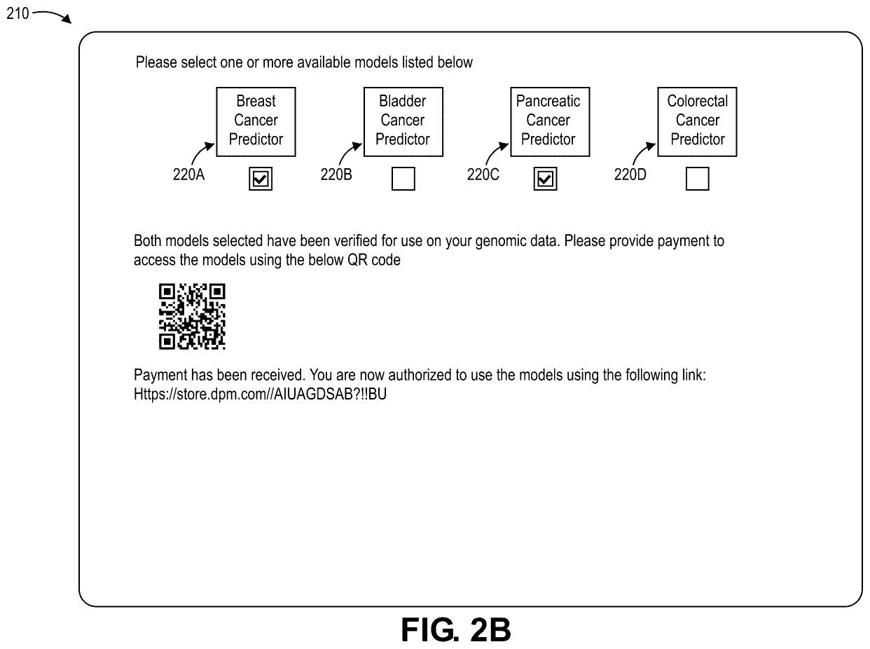

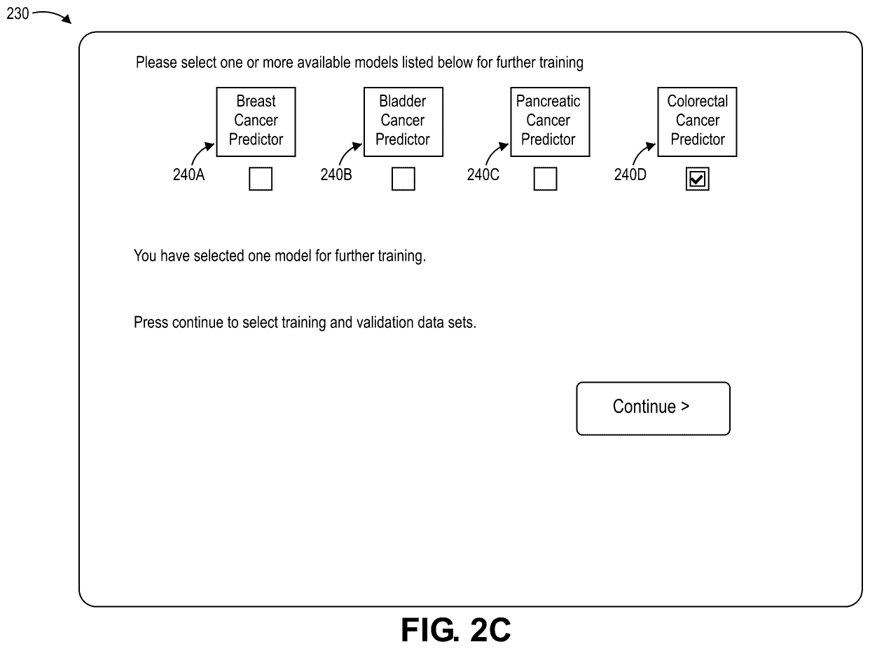

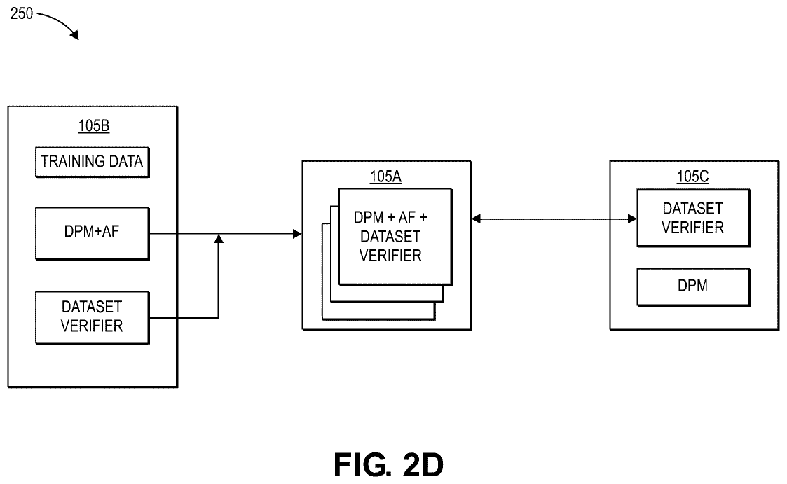

In some embodiments, the computing environment 100 may be and/or include a system for trustless sharing of DPMs 130. An exemplary system for trustless sharing of DPMs (TSD system) 200 is depicted in FIG. 2A, and may include the application store compute resource 105A, the application provider compute resource 105B, and/or the application consumer compute resource 105C, as previously described.

According to the TSD system 200, the application store compute resource 105A may receive or otherwise obtain (e.g., from the application provider compute resource 105B via network 110) one or more DPMs 130 trained to generate a prediction (e.g., a diagnostic prediction, a prognostic prediction, a recommendation, a risk assessment, etc.) based upon genomic data, such as a diagnostic prediction of a risk of cancer occurrence of a patient. The application store compute resource 105A may store the DPMs 130 in a memory, such as memory 124 and/or databases 126. The application store compute resource 105A may subsequently distribute the DPMs 130, e.g., to application consumers via application consumer compute resource 105C over network 110. In some aspects, the application provider compute resource 105B and the application store compute resource 105A may be one and the same, for example where the application provider hosts an online/digital store to sell their DPMs 130, in which case application store compute resource 105A and application provider compute resource 105B may be referred to interchangeably.

In at least some aspects of the TSD system 200, the application store compute resource 105A may further receive (e.g., via application provider compute resource 105B over network 110) adaptation factor(s) of a reference genomic dataset associated with a DPM 130, such as adaptation factors of a genomic training dataset used to train the DPM 130. The application store compute resource 105A may store the adaptation factors in a memory, such as memory 124, databases 126, and/or other suitable memory. As previously described, the adaptation factor(s) may be used to transform one genomic dataset to conform to the dataset-specific nature (e.g., biases, domain shifts, domain changes, covariate shifts, etc.) of another dataset. In one aspect, transforming one genomic dataset to conform to another may include transforming an application consumer genomic dataset to conform to a reference genomic dataset for which the DPM 130 is trained on, and/or may operate on, to generate accurate predictions. In one aspect, this may include transforming an application provider genomic dataset or a reference genomic dataset (e.g., for DPM 130 training) to conform to an application consumer genomic dataset.

For example, the reference genomic dataset may be generated by a first set of sequencing equipment and used by the application provider to train the DPM 130. The application consumer genomic dataset may be generated by a second set of sequencing equipment, and the respective genomic datasets of the application provider and consumer may have differences in characteristics (e.g., biases) due to their respective sets of sequencing equipment. The adaptation factor(s) of the training data of the DPM 130 may be used to transform (e.g., via the data transformers 134) the application consumer genomic dataset to conform to the application provider training genomic dataset, such that the transformed application consumer genomic dataset is compatible with the associated DPM 130.

In at least some aspects, the application store compute resource 105A may further receive (e.g., via application provider compute resource 105B over network 110) one or more data transformers 134. For example, the transformer 134 may transform an application consumer's genomic dataset to conform to the dataset-specific nature of a training dataset used to train the DPM 130 the consumer wishes to purchase. As previously indicated the combination of a DPM 130 and associated adaptation factors and/or data transformers 134 may be collectively referred to as a DPA.

In at least some aspects, the application store compute resource 105A may receive, e.g., from an application consumer via application consumer compute resource 105C, a request for one or more DPMs 130. In at least some aspects, the application store compute resource 105A may generate and/or provide for display a GUI, e.g., which the application consumer compute resource 105C displays for requesting one or more DPMs 130. The GUI may include one or more representations corresponding to one or more DPMs 130. The representations may be text, icons, checkboxes, or any other suitable representations displayable via a GUI, display, and/or other user interface. The representations may indicate DPMs 130 offered via the application store compute resource 105A to third parties, such as an application consumer. The GUI may be a GUI associated with the application store compute resource 105A, a GUI associated with the application consumer compute resource 105C, and/or any other suitable GUI.

FIG. 2B illustrates an exemplary GUI 210 for distributing four DPMs 130 represented by icons 220A-220D. The GUI 210 may be displayed on the screen of an application consumer compute resource 105C. Using the GUI 210, the DPM consumer indicates they would like to download and/or purchase the breast cancer predictor DPM and the pancreatic cancer predictor DPM by selecting the checkboxes associated with their icons 220A, 220C. For example, the consumer may use a mouse attached to the application consumer compute resource 105C via the I/O module 146 to select the icons 220A, 220C.

The application store compute resource 105A may provide and/or distribute to the application consumer compute resource 105C one or more of the requested DPMs 130. To distribute the DPMs 130, the application store compute resource 105A may offer a download of the DPMs 130 (e.g., provide a hyperlink to download the DPMs 130 to the application consumer compute resource 105C), may remove restrictions preventing use and/or access of the DPMs 130 by the application consumer (e.g., via a subscription, a payments, etc.), and/or provide the DPMs 130 in any other suitable manner.

As described above, in at least some aspects a consumer may transform (e.g., via the application consumer compute resource 105C) their own genomic dataset, and the DPM 130 is applied to the transformed consumer genomic dataset in the consumer's computing environment. In such an aspect, the DPM 130 may include one or more associated adaptation factors and/or a data transformers 134. The application consumer may use the adaptation factors and/or data transformers 134 associated with the DPM 130 to transform a target/application consumer genomic dataset to conform to the dataset-specific nature of the reference/training genomic dataset which is compatible and/or otherwise suitable for the DPM 130 without the application provider having to share their reference/training genomic data, nor the application consumer having to share their target/application consumer genomic data. As a result of transforming the target/application consumer genomic dataset, the application consumer may apply the DPM 130 to the transformed target/application consumer genomic dataset to generate a diagnostic prediction. If the DPM 130 was applied to non-transformed target/application consumer genomic dataset, the DPM 130 may be unable to generate a prediction, and/or generate an erroneous or otherwise inaccurate prediction.

In at least some aspects, the DPM model may be trained using training data which allows the DPM to operate on the consumer's unchanged genomic dataset, as described above. This may include fine-tuning an already-trained DPM to operate on the consumer's unchanged dataset. FIG. 2C illustrates an exemplary GUI 230 for training four DPMs 130, represented by icons 240A-240D, to operate on an unchanged consumer dataset. The GUI 230 may be displayed, for example, on the screen of an application provider compute resource 105B. Using the GUI 230, the DPM provider indicates they would like to train the colorectal cancer predictor DPM by selecting the checkbox associated with the icon 240D. For example, the application provider may use a mouse attached to the application provider compute resource 105B via the I/O module 146 to select the icon 240D. The DPM provider may then select the appropriate training data to train the DPM, such as training and validation genomic datasets. Once trained, the application store compute resource 105A may provide and/or distribute to the application consumer compute resource 105C one or more of the requested DPMs 130 trained to operate on the unchanged consumer genomic dataset. To distribute the DPMs 130, the application store compute resource 105A may offer a download of the DPMs 130 (e.g., provide a hyperlink to download the DPMs 130 to the application consumer compute resource 105C), may remove restrictions preventing use and/or access of the DPMs 130 by the application consumer (e.g., via a subscription, a payments, etc.), and/or provide the DPMs 130 in any other suitable manner.

Although distributing the one or more DPMs 130 to an application consumer has been described, the TSD system 200 may be used to distribute a plurality of DPMs 130 to a plurality of consumers. For example, the application store compute resource 105A may receive from a second application consumer, a second request and/or indication of a DPM 130. In response, the compute resource 105A may provide to the application consumer, verified DPMs 130.

### Distributing a Verifiable DPM

In at least some embodiments, the computing environment 100 may be a system for distributing verifiable DPMs 130. FIG. 2D illustrates an exemplary system for distributing verifiable DPMs (DVD system) 250. Similar to the TSD system 200, the DVD system 250 may include an application store compute resource 105A, an application provider compute resource 105B, and/or an application consumer compute resource 105C.

According to the DVD system 250, the application store compute resource 105A may receive or otherwise obtain (e.g., from the application provider compute resource 105B via network 110) one or more DPMs 130 (e.g., model 128) trained to generate a diagnostic prediction based upon genomic data, such as a diagnostic prediction of a risk of cancer occurrence. The application store compute resource 105A may store the DPMs 130 in a memory, such as memory 124 and/or databases 126. The application store compute resource 105A may subsequently distribute the DPMs 130, e.g., to application consumers via application consumer compute resource 105C over network 110. In some aspects, the application provider compute resource 105B and the application store compute resource 105A may be one and the same, for example where the app provider hosts an app store to sell their DPMs 130, in which case application store compute resource 105A and application provider compute resource 105B may be referred to interchangeably.

In at least some aspects of the DVD system 250, the application store compute resource 105A may receive (e.g., via application provider compute resource 105B over network 110) adaptation factor(s) of a reference genomic dataset associated with a DPM 130, such as a genomic training dataset of the DPM 130. In at least some aspects, the application store compute resource 105A may receive (e.g., via application provider compute resource 105B over network 110) one or more data transformers 134 associated with a DPM 130, as previously described. The application store compute resource 105A may store the adaptation factors and/or data transformer 134 in a memory, such as memory 124, databases 126, and/or other suitable memory.

In at least some aspects, the application store compute resource 105A may receive, e.g., from an application consumer via application consumer compute resource 105C, a selection and/or otherwise indication of a DPM 130 of the DPMs 130 indicated, such as the example of FIG. 2B. Before the application store compute resource 105A provides one or more DPMs 130 (e.g., to the application consumer), the DPM 130 (s) may be verified, e.g., based upon its suitability and/or compatibility for use with at least a cohort of a genomic dataset (e.g., application consumer genomic dataset) of a recipient of the DPMs 130. The verification of one or more indicated DPMs 130 may be provided by the dataset verifier 136, and may include determining a gene-outcome correlation or a gene-gene correlation between a source genomic dataset of the DPM 130 (e.g., training genomic dataset) and a target genomic dataset (e.g., dataset of the DPM 130 recipient/purchaser).

In at least some aspects, the dataset verifier 136 may be provided to a party requesting the DPM 130 (e.g., consumer) to verify an associated target genomic dataset, such as application store compute resource 105A providing the dataset verifier 136 to application consumer compute resource 105C. In such an aspect, the dataset verifier 136 may indicate verification of one or more DPMs 130 to the application store compute resource 105A, such as via a verified request for one or more verified DPMs 130. The verification may be and/or include a message, signal, an indication describing the verification, a metric, and/or other suitable verification.

In at least some aspects, the application store compute resource 105A may receive, e.g., from the application consumer compute resource 105C, information corresponding to an application consumer genomic dataset which will allow for verification of the application consumer genomic dataset. This may include a basis, a data descriptor and/or one or more correction factors. A data descriptor may describe the population the model was trained on, such as clinical characteristics (e.g., cancer type, stage, histopathology, age, therapies), species (e.g., animal, viral, human, organoid), or other suitable descriptors. In some aspects, the data descriptor may be a database query (e.g., an SQL query, a NoSQL query, etc.). The data descriptor may include instructions for selecting data of one or more subjects used in training (e.g., data products, data features, data characteristics, etc.), text describing output of the model (e.g., text describing one or more predictions), the domain of patients used in training, etc. In one example, features of the data may be analyzed to determine whether they align with the predictive task of the machine learning model. The features may be analyzed to determine whether they are related to a real-world scenario. The data may be analyzed to determine its distribution (e.g., skewness). The application store compute resource 105A may analyze the dimensionality of the data, the dispersion of the data and the distribution shape of the data. Correction factors may include a collection of mathematical constants used to normalize and/or transform data. Based upon the information, the application store compute resource 105A may verify a diagnostic prediction model, e.g., using the dataset verifier 136. Other suitable methods may be implemented for verifying a DPM 130.

In at least some aspects, the application store compute resource 105A may not receive, e.g., from an application consumer via application consumer compute resource 105C, a selection and/or otherwise indication of a DPM 130, but rather may receive only the information corresponding to the application consumer genomic dataset. In such an aspect, the application store compute resource 105A may verify via dataset verifier 136 one or more DPMs 130 which are suitable and/or compatible with the application consumer genomic dataset, and inform the user (e.g., via a GUI such as GUI 210) of the verified DPMs 130 they are allowed to purchase.

The application store compute resource 105A may authorize one or more DPMs 130 for distribution to the application consumer via the application consumer compute resource 105C. The compute resource 105A may provide authorization for the verified DPMs 130 upon the consumer enrolling in a subscription service to use any of the verified DPMs 130, upon receiving a payment for one or more individual DPMs 130 and/or any other suitable manner of authorizing a DPM 130. In at least some embodiments, authorization of a DPM 130 may not be required and/or necessary, and upon verification of a DPM 130, the compute resource 105A may provide the verified DPMs 130 to the application consumer.

Although distributing an authorized and/or verified DPM 130 to a single application consumer has been described, the DVD system 250 may be used to distribute a plurality of DPMs 130 to a plurality of consumers. For example, the application store compute resource 105A may receive from a second application consumer, a second request for a DPM 130 and/or information corresponding to a second application consumer genomic dataset. In response, the compute resource 105A may verify, authorize and provide to the application consumer, the verified DPM 130.

### Electronic Health Records

In at least some embodiments, the computing environment 100 may be and/or include an electronic health record (EHR) system. The EHR system may include an EHR client, such as application consumer compute resource 105C, and a remote computing device, such as compute resource 105, application store compute resource 105A or application provider compute resource 105B.

The EHR system may include a plug-in architecture to provide plug-ins to the EHR system. The plug-in architecture, for example, may include one or more APIs, microservices, applets, and/or other suitable add-on tools and the like, especially those which may enhance the operation and/or capabilities of a software program or executable-instructions of the EHR system. In at least some aspects, the plug-in architecture is an Epic architecture or an FHIR architecture.

The remote computing device may include one or more DPMs 130. The remote computing device may further include the data transformers 134 corresponding to at least one DPM 130 to transform the target genomic data into transformed genomic data compatible with a respective DPM 130, as previously described.

In at least some aspects, the EHR system may display a representation of one or more DPMs 130 via a GUI of the EHR client, such as the GUI illustrated in FIG. 2B. In at least some aspects, the GUI may only provide representations of DPMs 130 (e.g., icons) suitable for generating diagnostic predictions using the genomic data of EHRs of one or more patients, e.g., DPMs 130 may only be shown in the GUI when they are compatible with the patient's genomic data. For example, the EHR system may generate or otherwise provide genomic data for patients of the EHR system. In one aspect, the EHR patient genomic data may be produced by the same equipment, using the same methodology, etc., and as a result, have the same characteristics, such as format, fields, batch effects or population/domain changes, biases, and/or other suitable characteristics. In one aspect, the genomic data may vary from patient to patient with respect to its characteristics. In either case, the application store compute resource 105A may be able to provide DPMs for distribution to make a prediction from the EHR patient genomic dataset (e.g., a prognostic prediction, a diagnostic prediction, etc.) based upon data transformation via the data transformer 134, verifying the genomic data via the dataset verifier 136, etc.

The EHR client may receive a selection of at least one of the DPMs 130 via the GUI, such as from a user of application consumer compute resource 105C. The EHR system may generate, at the EHR client, an electronic request including (i) an indication of the selected DPMs 130 and (ii) one or more EHRs of a patient including at least some genomic data. For example, a healthcare provider using an EHR client may use a mouse to select icons associated with one or more DPMs 130 to generate predictions for a patient based upon the patient's genomic data in their EHR.

The EHR client may transmit, via an electronic network such as network 110, the electronic request to the remote computing device. The remote computing device may apply the indicated DPMs 130 to the genomic data of one or more EHRs of patients.

The EHR client may receive from the remote computing device an electronic result including one or more diagnostic predictions generated by the selected diagnostic prediction models, and display the electronic result via the GUI of the EHR client.

### Transferability of a Prognosticator

In at least some embodiments, the computing environment 100 may determine whether a model 130 such as a prognosticator model is transferable by determining whether risk genes and protective genes in different genomic datasets (e.g., an application provider genomic dataset and an application consumer genomic dataset) have associated outcomes. If the results of the test are statistically significant, the prognosticator model may be successfully transferred.

In such an embodiment, a compute resource such as compute resource 105, 105A may process an application provider genomic dataset using the prognosticator model to generate an application provider cohort response association outcome, e.g., a gene-outcome correlation of a cohort of the application provider genomic dataset. The compute resource may process the application consumer genomic dataset using the prognosticator model to generate an application consumer cohort response association outcome, e.g., a gene-outcome correlation of a cohort of the application consumer genomic dataset. The compute resource may generate a statistical significance between the application provider cohort response association outcome and the application consumer cohort response association outcome. When the statistical significance is highly significant (e.g., a statistical indicator indicates whether an algorithm can be applied to datasets having a new domain), the compute resource may determine that the prognosticator model is transferable between the application provider genomic dataset and the application consumer genomic dataset. Significance measures that may be applied include associated weights and feature importance, gene enrichment, differential expression, over representation, etc.

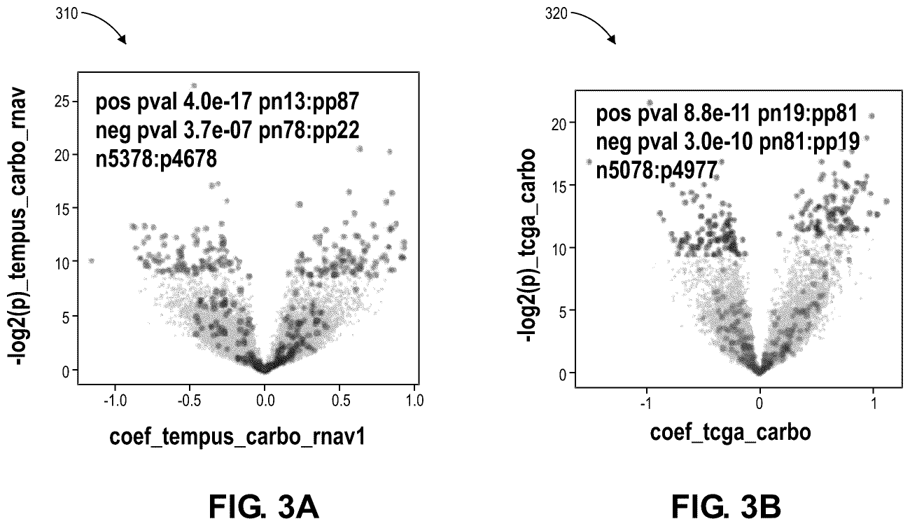

FIGS. 3A and 3B depict exemplary graphs 310, 320 of molecular reproducibility results of gene-association correlations for responses to carboplatin in Endometrioid cancers of an application provider genomic dataset and application consumer dataset respectively. Risk genes and protective genes that were found in a first genomic dataset (private dataset) of an application provider according to graph 310 of FIG. 3A are tested to indicate whether they have the same association with outcomes in an application consumer genomic dataset (TCGA dataset) according to graph 320 of FIG. 3B. The graphs 310, 320 illustrate that the p-values are highly significant, confirming the model trained with these genes prognosticates both cohorts of the application provider genomic dataset of graph 310 and application consumer genomic dataset of graph 320 without requiring data sharing between the genomic provider or consumer. In some aspects, other types of testing and quantification may be included in the present techniques, including in irreproducible discovery rate (IDR) test, a gene set enrichment test, gene set enrichment analysis (GSEA) pre-rank, and/or other enrichment, similarity, dissimilarity tests.

The molecular reproducibility results indicate whether a prognosticator is transferable between two data sources (e.g., a proprietary source and TCGA). The present techniques include performing a gene enrichment test by testing that risk genes and protective genes that were found in the proprietary model also have the same association with outcomes in TCGA. Based on this experiment indicating that a test is statistically significant, the present techniques are able to transfer a predictor from one dataset to another. FIGS. 3A and 3B depict testing genes for association with response to carboplatin in Endometrioid cancers. These figures depict that p-values are highly significant, and indeed the model trained with these genes prognosticates both cohorts. The test does not require patient-level data sharing. The p-value significance threshold may be above 0.05, 0.1, 0.15, 0.2, etc. In some aspects, the p-value significance threshold may be above 0.8, 0.85, 0.9, etc. An exemplary p-value threshold may be 0.05.

If gene and associated outcomes are not statistically significant, gene-gene correlations of the training and validation datasets of the prognosticator model may be evaluated. A p-value threshold of insignificance may be below 0.7, 0.65, 0.6, etc. In some aspects, a p-value threshold of insignificance may be below 0.3, 0.25, etc. In such an embodiment, the computer environment 100 may receive a second genomic dataset, the reference genomic dataset used to train the prognosticator model, and one or more validation genomic datasets used to validate the prognosticator model. The compute resource may generate a plurality of top genes correlated with the reference genomic dataset and the second genomic dataset and generate an associated result vector, as illustrated in FIG. 3B. For each validation genomic dataset of the one or more validation genomic datasets, the compute resource may generate a plurality of reference correlation values by processing the reference genomic dataset and the plurality of top genes; generate a respective plurality of validation correlation values by processing the each validation genomic dataset and the plurality of top genes; generate respective cosine similarity values between (i) each of the respective plurality of reference correlation values, and (ii) each of the respective plurality of validation correlation values; store the respective cosine similar values in the result vector; and cause the result vector to be transmitted to a remote device via the electronic network.

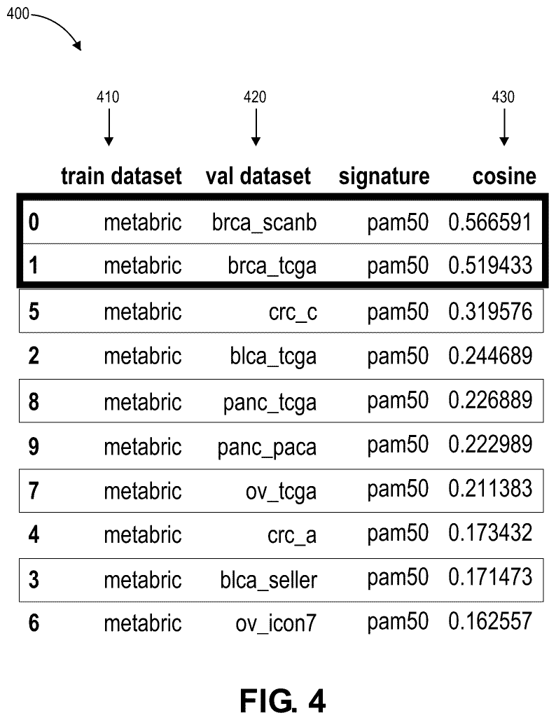

FIG. 4 depicts an exemplary table 400 of gene-gene correlation results for training genomic datasets of a first column 410 and validation genomic datasets of a second column 420 of a PAM50 breast cancer subtyping model. A public (breast) training genomic dataset is used to calibrate gene-gene correlations of the PAM50 breast cancer subtyping model. Validation genomic datasets of the second column 420 are assessed for similarity in their gene-gene covariance structures, depicted in the cosine column 430. The algorithm correctly ranked SCANB, a Swedish breast cancer clinical trial dataset, and BRCA-TCGA, the TCGA breast cancer datasets as top results for similarity in their gene-gene covariance structures.

An evaluation of the similarity between a training dataset, a set of validation datasets, and a gene signature may generate the table 400, wherein the evaluation may include: (i) selecting a set of top k genes that are most highly correlated with the gene signature on the training dataset; (ii) iterating through the validation datasets and for each, calculating two correlation matrices for the training and validation dataset of the gene signature and top k genes; (iii) calculating the cosine similarity between those two correlation matrices; and (iv) ranking the validation datasets based on the cosine similarity and select the highest similarity dataset.

FIG. 4 depicts training data that was used for learning different signatures. Top left, Metabric (breast) dataset was used to calibrate gene-gene correlations of the PAM50 breast cancer subtyper. Other datasets were assessed for similarity in their gene-gene covariance structures are depicted in the cosine column. The present techniques correctly top rank SCANB, a Swedish breast cancer clinical trial dataset, and BRCA-TCGA, the TCGA breast cancer dataset.

### Verifying a Genomic Dataset for a Diagnostic Prediction Model

In at least some embodiments, the computing environment 100 may automatically verify (e.g., via the dataset verifier 136) a genomic dataset for use with a diagnostic prediction application. In such an embodiment, a compute resource, such as compute resources 105, 105A, 105B, may apply the dataset verifier to one or more genomic datasets to determine a gene-outcome correlation expressed by each genomic dataset. The dataset verifier may determine whether the gene-outcome correlation expressed by one genomic dataset is substantially similar to the gene-outcome correlation expressed by the one or more other genomic datasets. If the respective gene-outcome correlations of different genomic datasets are substantially similar, the compute resource may generate a gene-outcome validation dataset indicating the substantial similarities. In some aspects, gene-to-label associations in a plurality of datasets may be assessed. For example, a test of intersections between top N ranked genes (e.g., a Fisher exact test, a chi-squared test, etc.) may be used to determine an association for a given number N. For example, N may be set to equal 100. The intersection test may be performed for different values of N and the dataset that gives the best similarity may be selected.

If the respective gene-outcome correlations are not substantially similar, the compute resource may apply the algorithm (e.g., via the dataset verifier 136) to the at least one application consumer genomic dataset which is not substantially similar to determine gene-gene correlations, and generate a gene-gene validation dataset. The gene-gene validation dataset may indicate the covariance between at least one of the application consumer genomic datasets which are not substantially similar to a respective application provider genomic dataset.

### Finding Sub-Populations Responsive to a Drug

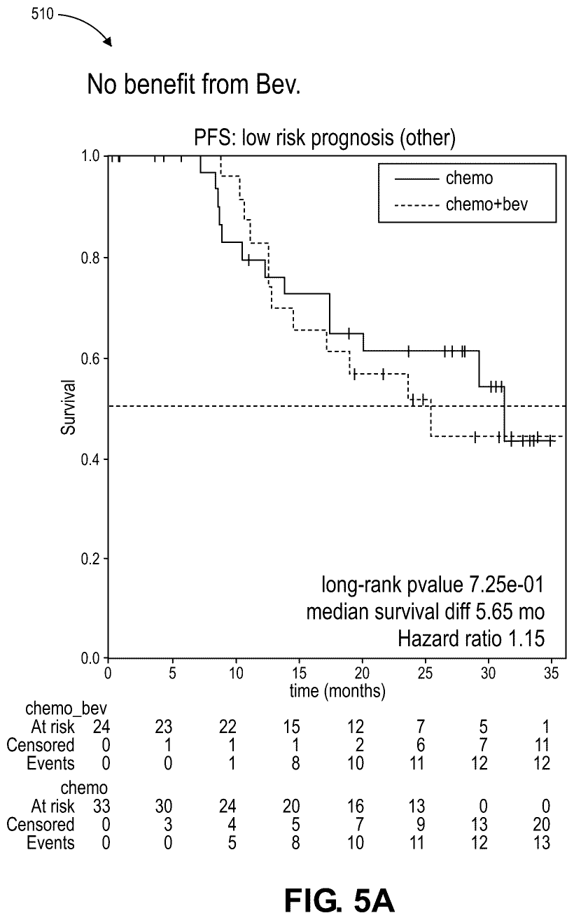

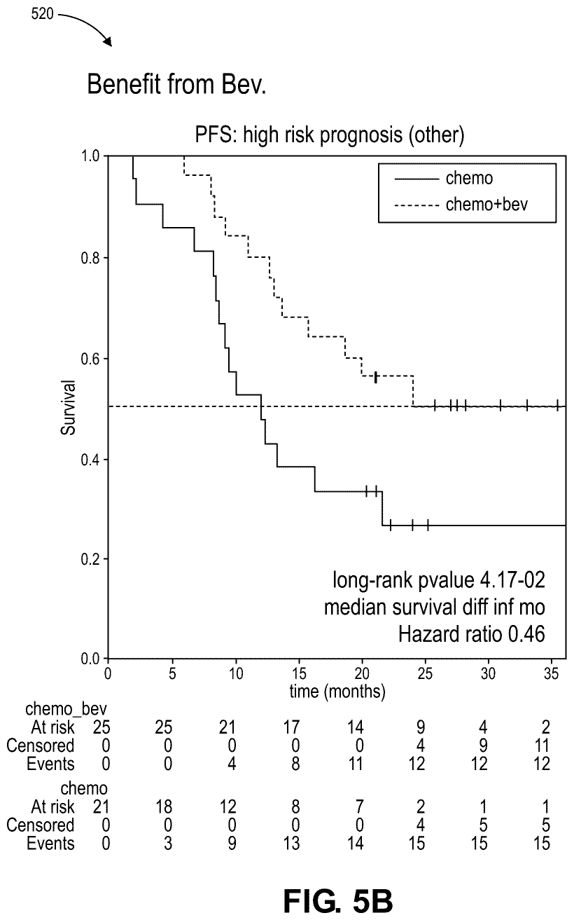

In one embodiment, the computing environment 100 may rescue a failed drug trial. In such an embodiment, the compute resource such as compute resources 105, 105A may receive a DPM such as DPM 130 trained to prognosticate a genomic condition based upon a genomic dataset. The compute resource may apply the DPM to a historical genomic dataset indicative of a failed trial to generate a prognostication of the genomic condition. The historical genomic dataset may be stored in memory 124, store in database 126, obtained from a third party (e.g., shared, purchased, publicly available), or otherwise available to the DPM. The compute resource may generate one or more reports indicating the prognostication of the genomic condition. FIGS. 5A and 5B illustrate an exemplary graph 510 of results for applying a prognosticator model TCGA to a training genomic dataset, and an exemplary graph 520 for applying the prognosticator model to results drug trial genomic dataset respectively, according to some aspects.

FIGS. 5A and 5B depict examples of rescuing subpopulations. For example, a proprietary ovarian cancer prognosticator and a drug response predictor could be provided, allowing a company that produces that drug to rescue a subpopulation that did not respond to the drug in a trial. The present techniques may include training an Ovarian cancer prognosticator using TCGA data, and calculating a TCGA RNA-seq data transformer. The present techniques may also include predicting (e.g., on the NEJM Bevacizumab trial) in ovarian cancer using microarray data. In this example, the prognosticator was found to be Avastin response predictive. The present techniques may include training a prognosticator on TCGA. While training, the predictor may not be aware of its future task of prognosticating (e.g., a Avastin/Bevacizumab clinical trial that was run by Genentech). Testing the prognosticator on a predictive task shows prognostic and predictive ability on the trial (right figure), potentially identifying a new CDX.

### Exemplary Computer-Implemented Methods

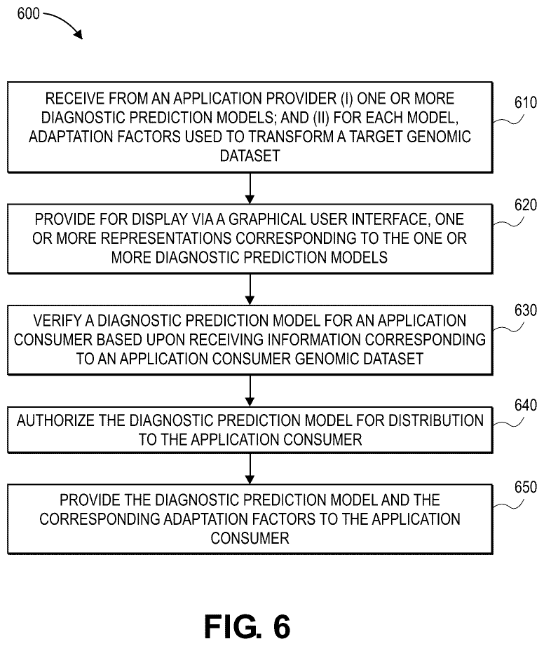

FIG. 6 depicts a flow diagram of an example computer-implemented method 600 for distributing diagnostic prediction models. The computer-implemented method 600 may operate as a stand-alone method, may operate in conjunction with other systems and/or methods described herein, and/or may operate in conjunction with embodiments of at least a portion of the computing environment 100 of FIG. 1, and/or of any one or more components and/or devices related thereto, and/or with other systems, processors, databases and/or devices.

For example, in some embodiments, the processor 120 may execute at least a portion of the computer-implemented method 600 in conjunction with one or more other components of the compute resource 105, such as the DPM 130, the data transformers 134, the dataset verifier 136, etc. The computer-implemented method 600 may include additional or alternate steps other than those described with respect to FIG. 6, in embodiments.

Generally speaking, the computer-implemented method 600 may include receiving, by one or more processors, from an application provider (i) one or more diagnostic prediction models, each model trained to generate a respective diagnostic prediction based upon genomic data; and (ii) for the each model, adaptation factors used to transform a target genomic dataset to conform to a dataset-specific nature of a reference genomic dataset of the each model (block 610). The diagnostic prediction model may be at least one of a diagnosticator model, a prognosticator model, a subtyping model, or a predictive model.

The computer-implemented method 600 may include providing, by the one or more processors for display via a graphical user interface, one or more representations corresponding to the one or more diagnostic prediction models (block 620).

The computer-implemented method 600 may include verifying, by the one or more processors, a diagnostic prediction model of the one or more diagnostic prediction models for an application consumer based upon receiving information corresponding to an application consumer genomic dataset (block 630). In at least some aspects, both of the application provider and the application consumer may be affiliated with a biotechnology entity, an educational entity, a collaborator of a provider of the diagnostic prediction models, or a pharmaceutical entity. In at least some aspects, the reference genomic dataset may be generated by a first set of sequencing equipment and the application consumer genomic dataset may be generated by a second set of sequencing equipment. The reference genomic dataset and the application consumer genomic dataset may have differences in characteristics due to their respective sets of sequencing equipment. In some aspects, the reference genomic dataset may be a public dataset or laboratory-specific dataset, and the application consumer genomic dataset may be a public dataset or laboratory-specific dataset.

The computer-implemented method 600 may include authorizing, by the one or more processors, the diagnostic prediction model for distribution to the application consumer (block 640).

The computer-implemented method 600 may include providing, by the one or more processors, the diagnostic prediction model and the corresponding adaptation factors to the application consumer (block 650).

In at least some aspects, the computer-implemented method 600 may include determining, by the one or more processors, at least one relevant label in common, between the application consumer genomic dataset and the reference genomic dataset, for a gene and associated outcome. The gene and associated outcome may be based upon risk genes and protective genes.

In at least some aspects, the computer-implemented method 600 may include determining, by the one or more processors, pairs of genes which have at least one covariance between the application consumer genomic dataset and the reference genomic dataset. In at least some aspects, determining the pairs of genes which have at least one covariance may include determining, by the one or more processors, at least one relevant label is not in common, between the application consumer genomic dataset and the reference genomic dataset, for a gene and associated outcome.

In at least some aspects, the computer-implemented method 600 may include generating, by the one or more processors, a cosine similarity between the application consumer genomic dataset and the reference genomic dataset.

In at least some aspects, the computer-implemented method 600 may include (i) verifying, by the one or more processors, the diagnostic prediction model of the one or more diagnostic prediction models for a second application consumer based upon receiving information corresponding to a second application consumer genomic dataset; (ii) authorizing, by the one or more processors, the diagnostic prediction model for distribution to the second application consumer; and (iii) providing, by the one or more processors, the diagnostic prediction model and the corresponding adaptation factors to the second application consumer.

It should be understood that not all blocks of the exemplary flow diagram are required to be performed. Moreover, the block(s) from the exemplary flowchart may be performed in any particular implementation.

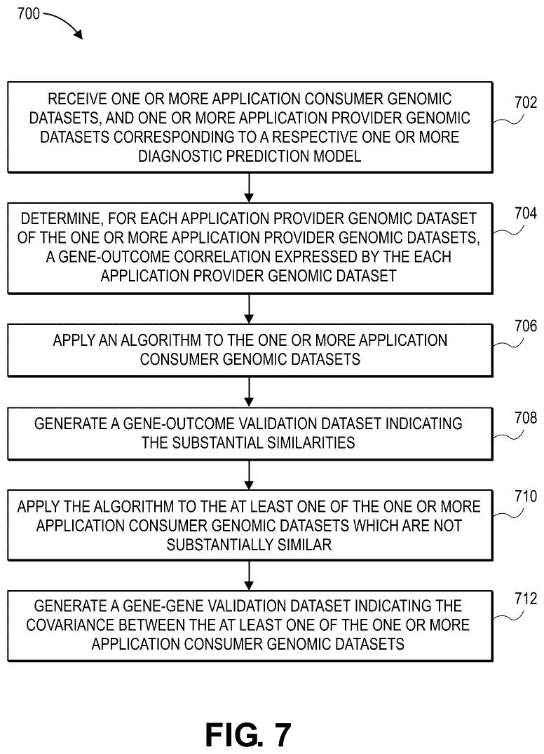

FIG. 7 depicts a flow diagram of an exemplary computer-implemented method 700 for automatically verifying a genomic dataset for use with a diagnostic prediction application. For example, before the diagnostic models are distributed, as in FIG. 2D, the verification procedures of method 700 may be executed (e.g., by the compute resource 105, the application store compute resource 105A, the application provider compute resource 105B and/or the application consumer compute resource 105C).

The method 700 may include receiving one or more application consumer genomic datasets, and one or more application provider genomic datasets corresponding to a respective one or more diagnostic prediction models (block 702). For example, in some aspects the memory 124 may receive the datasets via the processors 120, which receive the datasets via the network 110. In some aspects, the datasets may be from open source databases or proprietary databases/data sources owned/controlled by third parties. In other aspects, the datasets may be received via the databases 126 of FIG. 1 and may be owned/controlled by the proprietor of the compute resource 105. Herein, an application provider generally refers to a company or organization that wants to provide a model and data factors for use by others. Thus, the method 700 may receive one or more models and one or more data factors.

As discussed, transferring molecular models between proprietary labs or between a proprietary lab and another party is hindered by batch effects and/or effects caused by biological variability between different cancers and/or demographic variability between different subjects (e.g., age, gender, ethnicity, geography, etc.). For example, a cohort composition trained using colon cancer data may be verified using pancreatic cancer data. Population-level factors may also hinder model generalizability, such as socioeconomic factors, environmental factors, and healthcare access factors. Current batch effect algorithms correct batch effects by using patient level information. Thus, correcting one lab's data to another to allow a predictor to be applied from one lab to another requires a broker to facilitate a trustless transaction. The method 700 overcomes this limitation. For example, the method 700 may include determining, for each application provider genomic dataset of the one or more application provider genomic datasets, a gene-outcome correlation expressed by the each application provider genomic dataset (block 704).

The method 700 may include applying an algorithm to the one or more application consumer genomic datasets to determine, for each application consumer genomic dataset of the one or more application consumer genomic datasets, whether the gene-outcome correlation expressed by the each application provider genomic dataset is substantially similar to the gene-outcome correlation expressed by the one or more application consumer genomic datasets, wherein the gene-outcome correlation expressed by at least one of the one or more application consumer genomic datasets is substantially similar to the gene-outcome correlation expressed by at least one of the one or more application consumer genomic datasets (block 706). Herein, one or more application consumers may receive the consumer genomic datasets including respective model and data factors. This enables the application consumers to use the model and data factors that were developed by the application consumer, without sharing patient level information.

The method 700 may include generating a gene-outcome validation dataset indicating the substantial similarities, wherein the gene-outcome correlation expressed by at least one of the one or more application consumer genomic datasets is not substantially similar to the gene-outcome correlation expressed by at least one of the one or more application provider genomic datasets (block 708).

The method 700 may include applying the algorithm to the at least one of the one or more application consumer genomic datasets which are not substantially similar and the one or more application provider genomic datasets to determine pairs of genes which have at least one covariance between the at least one of the one or more application consumer genomic datasets which are not substantially similar and a respective application provider genomic dataset (block 710).

The method 700 may include generating a gene-gene validation dataset indicating the covariance between the at least one of the one or more application consumer genomic datasets which are not substantially similar to a respective application provider genomic dataset (block 712).

In some aspects, the gene-gene validation dataset indicates a cosine similarity between the at least one of the one or more application consumer genomic datasets which are not substantially similar and the respective application provider genomic dataset.

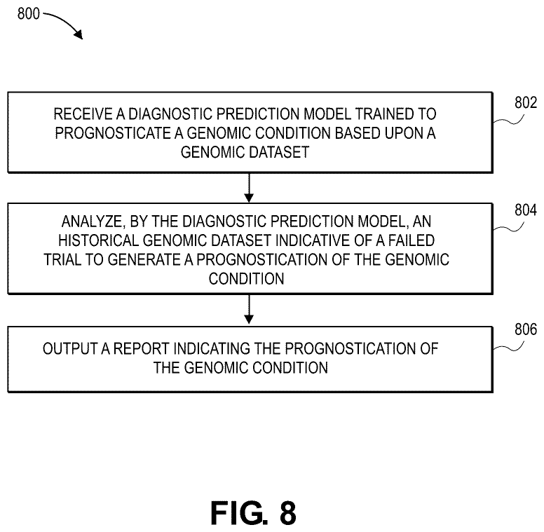

FIG. 8 depicts a flow diagram of an exemplary computer-implemented method 800 for rescuing failed drugs, according to some aspects. The method 800 may include receiving a diagnostic prediction model trained to prognosticate a genomic condition based upon a genomic dataset (802); analyzing, by the diagnostic prediction model, an historical genomic dataset indicative of a failed trial to generate a prognostication of the genomic condition (block 804); and outputting a report indicating the prognostication of the genomic condition (block 806).

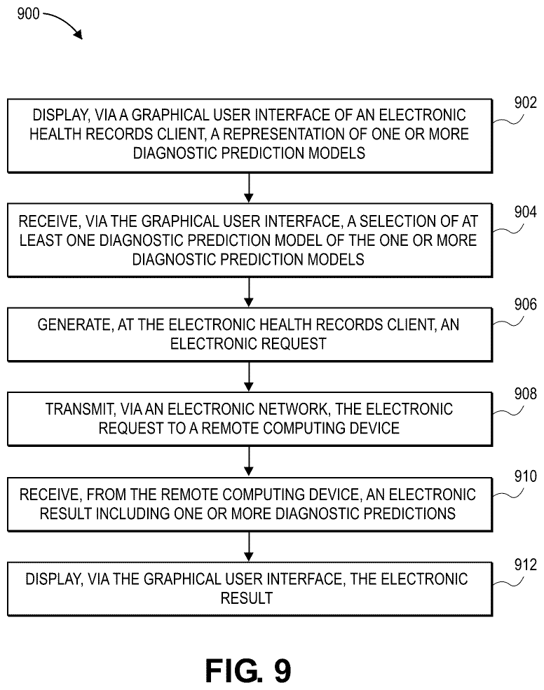

FIG. 9 depicts a flow diagram of an exemplary computer-implemented method 900 for providing a plugin to an electronic health record system via a plug-in architecture of the electronic health record system, according to some aspects. The method 900 may include displaying, via a graphical user interface of an electronic health records client, a representation of one or more diagnostic prediction models, each trained to generate a respective diagnostic prediction based upon genomic data of an electronic health record (block 902).

The method 900 may include receiving, via the graphical user interface, a selection of at least one diagnostic prediction model of the one or more diagnostic prediction models (block 904).

The method 900 may include generating, at the electronic health records client, an electronic request including (i) an indication of the at least one diagnostic prediction model selected and (ii) one or more electronic health records of a patient including at least some genomic data (block 906).

The method 900 may include transmitting, via an electronic network, the electronic request to a remote computing device (block 908).

The method 900 may include receiving, from the remote computing device, an electronic result including one or more diagnostic predictions generated by the at least one diagnostic prediction model (block 910).

The method 900 may include displaying, via the graphical user interface, the electronic result (block 912).

### Exemplary Model Exchange Environment Aspects

In some aspects, a technique referred to as SpinAdapt, described in U.S. patent application Ser. No. 17/548,084, entitled “Systems and Methods for Homogenization of Disparate Datasets,” filed on Dec. 10, 2021, and incorporated herein by reference in its entirety for all purposes, may be used to correlate model provider data with model consumer data.

In particular, the model provider may provide a SpinAdapt-compatible model and a predictive model to the compute resource 105. The compute resource 105 may provide these two models to the consumer (e.g., a purchaser). In this manner, the model provider and model consumer never exchange any information directly, and no patient-level information is shared. The SpinAdapt algorithm, for example, may fit target training data of the model provider to the SpinAdapt-compatible model and to the predictive model (e.g., by applying a learning algorithm to the target training data). The consumer may use the SpinAdapt-compatible model and the predictive model directly, by applying the SpinAdapt-compatible model to fit internal source data, applying a translator to the internal source data to generate corrected source data, and then applying the predictive model to the corrected source data to generate internal predictions. In some aspects, techniques other than SpinAdapt may be used to generate the corrected source data.

The consumer has the ability to verify that models are compatible with their internal source data, using a gene-gene correlation technique (generally for univariate data using unsupervised techniques to verify pairs of genes that have the same relationships in training data of the producer and client data of the consumer); and/or using gene-outcome correlations (generally for univariate data using supervised validation to confirm the signal and the predictor). For example, prior to purchasing any models, a client/consumer may download one or more dataset verifiers, test them on their internal source data (e.g., one or more cohorts) and request apps (e.g., models, predictors, data transformers, dataset verifiers, etc.) that are positively verified. In general, the dataset verifier for a particular model is provided to the app store (e.g., the compute resource 105) from the owner/trainer of the SpinAdapt-compatible/predictive model.

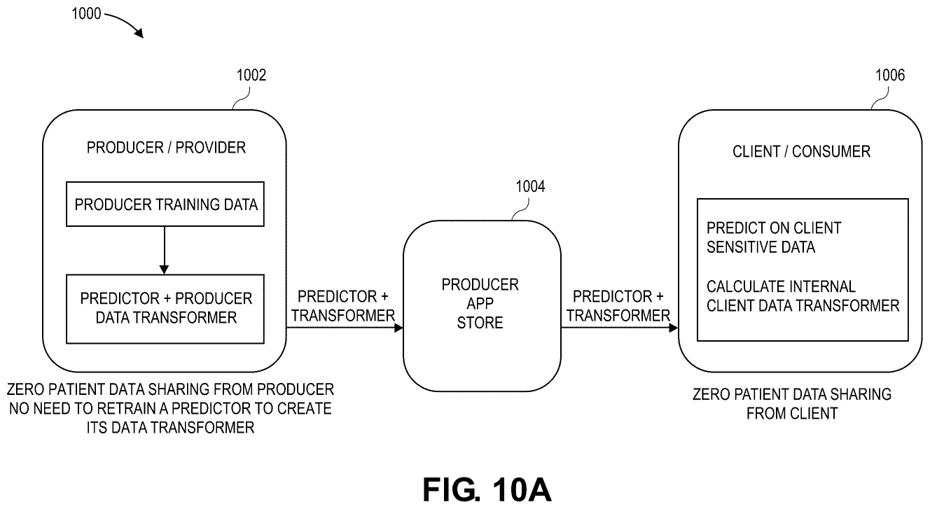

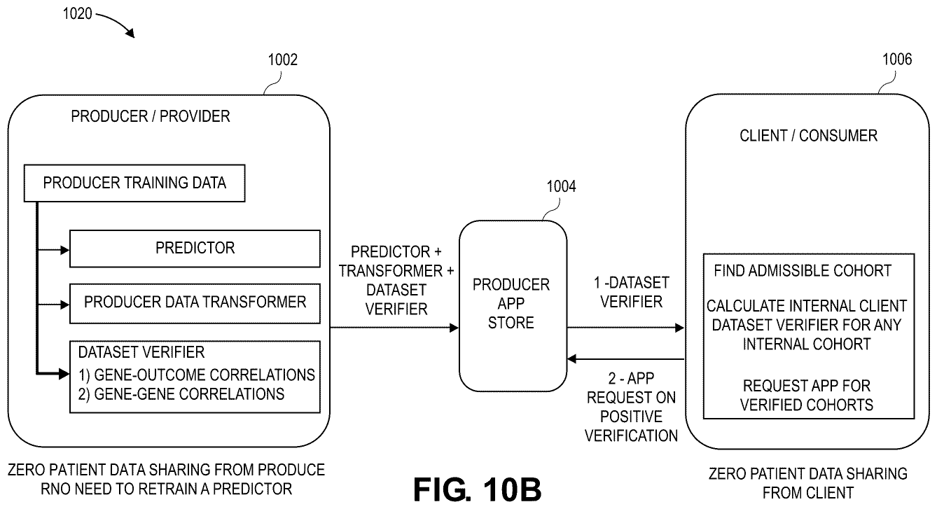

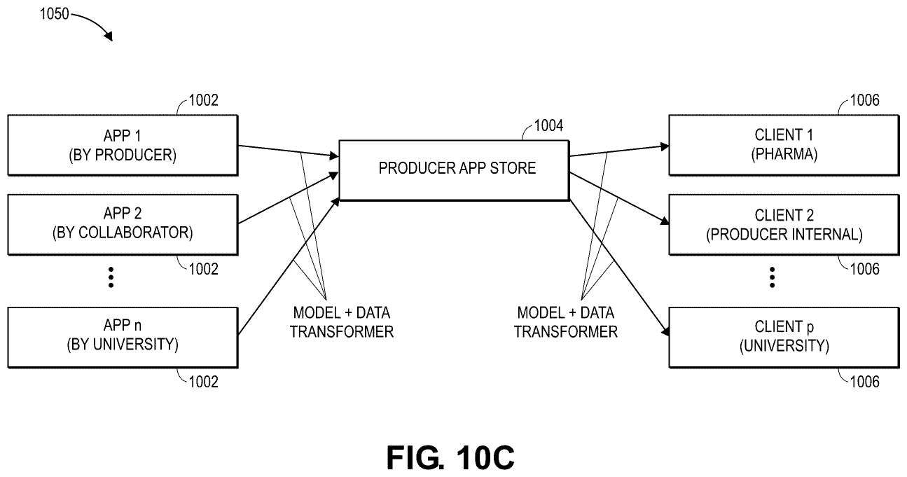

FIG. 10A depicts an exemplary model exchange environment 1000, according to some aspects. The exemplary model exchange environment 1000 may include a producer 1002, an app store 1004 and a consumer 1006. The producer 1002 may be the component that provides a predictor and a data transformer (e.g., the SpinAdapt components described above to the app store 1004. For example, the producer 1002, the app store 1004, and the consumer 1006 of FIG. 10A may correspond, respectively, to the application store application provider compute resource 105B of FIG. 1, the application store compute resource 105A of FIG. 1, and the application consumer compute resource 105C of FIG. 1.

FIG. 10B depicts an exemplary model exchange environment 1020 of FIG. 10A, including a dataset verifier generated at the producer 1002 and shared with the app store 1004, that the consumer 1006 may access to automatically verify which model(s) provided by the producer 1002 to the app store 1004 will work with the internal source data of the consumer 1006. In this manner, the consumer 1006 may determine which apps available at the app store 1004 will function with the internal source data of the consumer 1006 prior to purchase. It should be appreciated that here, the term “purchase”, “purchasing”, etc. should be read to include outright purchase, purchase of a perpetual license, purchase of a time-limited license, a short term rental, etc. Further, the producer 1002 may provide models that are limited in functionality (e.g., a demonstration application or model) that the consumer 1006 may pay to unlock, not unlike an in-application purchase on a modern mobile device platform such as the Google Play store, the Apple Store, etc. In general, the present techniques may be implemented in any suitable programming language, or a combination thereof (e.g., including R, Python, Swift, JavaScript, etc.).

FIG. 10C depicts an exemplary model exchange environment 1050, scaled to many consumer and producer participants, according to some aspects. The exemplary model exchange environment 1050 may correspond to the exemplary model exchange environments 1000, 1020 of FIGS. 10A-10B, in some aspects. The exemplary model exchange environment 1050 may include a plurality of producers 1002, each having one or more respective apps that include models and data transformers that are provided to the app store 1004. The app store 1004 may provide the one or more models and data transformers to a plurality of consumers 1006. It should be appreciated that in some aspects, one or more of the consumers 1006 may be owned/controlled by the proprietor of the producer 1002.

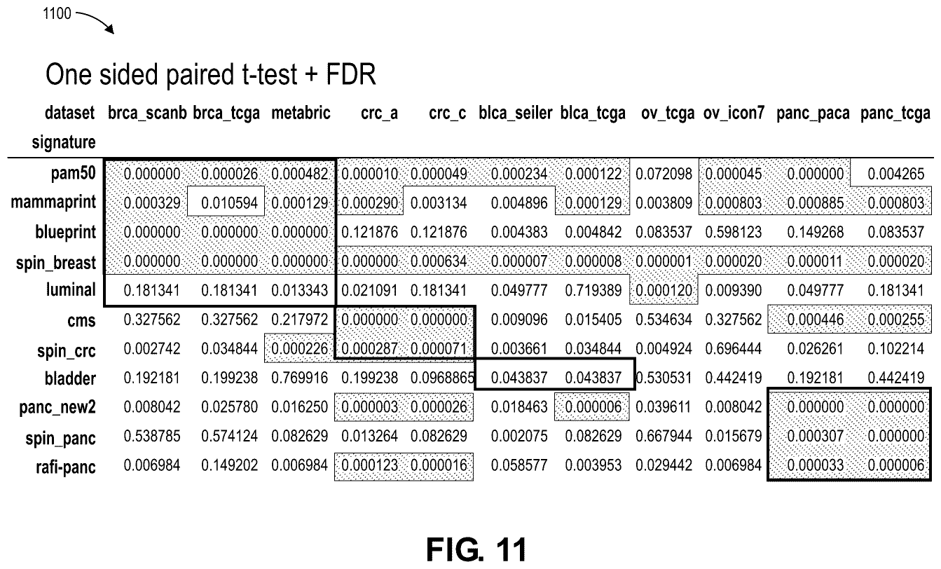

FIG. 11 depicts an exemplary cosine similarity matrix 1100 to determine whether a cohort of a genomic dataset is compatible with a DPM. The example matrix 1100 illustrates eleven columns of genomic datasets with varying cancer types, and eleven rows of genomic signatures that are either used in subtyping or prognostication. The matrix 1100 may indicate similarity between a genomic signature and a cohort of a genomic dataset using cosine similarities. The matrix 1100 is used to compute a cosine similarity between the signatures and datasets. In at least some embodiments, a similarity matrix may be populated with correlation coefficients, wherein the closer a number is to 1, the stronger the correlation. In some embodiments, a spectral norm is compared to the eigenvalue spectra of the matrices.

### EXEMPLARY ASPECTS

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

Aspects of the techniques described in the present disclosure may include any of the following aspects, either alone or in combination:

1. A computing system for distributing diagnostic prediction models comprising: one or more memories having stored thereon computer-executable instructions that, when executed by one or more processors, cause the computing system to: receive from an application provider: one or more diagnostic prediction models, each model trained to generate a respective diagnostic prediction based upon genomic data; and for the each model, adaptation factors used to transform a target genomic dataset to conform to a dataset-specific nature of a reference genomic dataset of the each model; provide for display via a graphical user interface, one or more representations corresponding to the one or more diagnostic prediction models; verify a diagnostic prediction model of the one or more diagnostic prediction models for an application consumer based upon receiving information corresponding to an application consumer genomic dataset; authorize the diagnostic prediction model for distribution to the application consumer; and provide the diagnostic prediction model and the corresponding adaptation factors to the application consumer.

2. The computing system according to aspect 1, the one or more memories having stored thereon instructions that, when executed, cause the computing system further to: determine at least one relevant label in common, between the application consumer genomic dataset and the reference genomic dataset, for a gene and associated outcome.

3. The computing system according to aspect claim 2, wherein the gene and associated outcome is based upon risk genes and protective genes.

4. The computing system according to any one of the preceding aspects, the one or more memories having stored thereon instructions that, when executed, cause the computing system further to: determine pairs of genes which have at least one covariance between the application consumer genomic dataset and the reference genomic dataset.

5. The computing system according to aspect 4, wherein to determine pairs of genes which have at least one covariance, the one or more memories have stored thereon instructions that, when executed, cause the computing system further to: determine at least one relevant label is not in common, between the application consumer genomic dataset and the reference genomic dataset, for a gene and associated outcome.

6. The computing system according to aspect 4, the one or more memories having stored thereon instructions that, when executed, cause the computing system to: generate a cosine similarity between the application consumer genomic dataset and the reference genomic dataset.

7. The computing system according to any one of the preceding aspects, the one or more memories having stored thereon instructions that, when executed, cause the computing system further to: verify the diagnostic prediction model of the one or more diagnostic prediction models for a second application consumer based upon receiving information corresponding to a second application consumer genomic dataset; authorize the diagnostic prediction model for distribution to the second application consumer; and provide the diagnostic prediction model and the corresponding adaptation factors to the second application consumer.

8. The computing system according to any one of the preceding aspects, wherein one or both of the application provider and the application consumer are affiliated with a biotechnology entity, an educational entity, a collaborator of a provider of the diagnostic prediction models, or a pharmaceutical entity.

9. The computing system according to any one of the preceding aspects, wherein the reference genomic dataset is generated by a first set of sequencing equipment and the application consumer genomic dataset is generated by a second set of sequencing equipment.

10. The computing system according to aspect 9, wherein the reference genomic dataset and the application consumer genomic dataset have differences in characteristics due to their respective sets of sequencing equipment.

11. The computing system according to any one of the preceding aspects, wherein the reference genomic dataset is a public dataset or a laboratory-specific dataset, and the application consumer genomic dataset is the public dataset or the laboratory-specific dataset.

12. The computing system according to any one of the preceding aspects, wherein the diagnostic prediction model is at least one of a diagnosticator model, a prognosticator model, a subtyping model, or a predictive model.

13. A computer-implemented method for distributing diagnostic prediction models comprising: receiving, via one or more processors, from an application provider: one or more diagnostic prediction models, each model trained to generate a respective diagnostic prediction based upon genomic data; and for the each model, adaptation factors used to transform a target genomic dataset to conform to a dataset-specific nature of a reference genomic dataset of the each model; providing, by the one or more processors for display via a graphical user interface, one or more representations corresponding to the one or more diagnostic prediction models; verifying, by the one or more processors, a diagnostic prediction model of the one or more diagnostic prediction models for an application consumer based upon receiving information corresponding to an application consumer genomic dataset; authorizing, by the one or more processors, the diagnostic prediction model for distribution to the application consumer; and providing, by the one or more processors, the diagnostic prediction model and the corresponding adaptation factors to the application consumer.

14. The computer-implemented method according to aspect 13, further comprising: determining, by the one or more processors, at least one relevant label in common, between the application consumer genomic dataset and the reference genomic dataset, for a gene and associated outcome.

15. The computer-implemented method according to aspect 14, wherein the gene and associated outcome is based upon risk genes and protective genes.

16. The computer-implemented method according to any one of aspects 13 to 15, further comprising: determining, by the one or more processors, pairs of genes which have at least one covariance between the application consumer genomic dataset and the reference genomic dataset.

17. The computer-implemented method according to aspect 16, wherein determining the pairs of genes which have at least one covariance further comprises: determining, by the one or more processors, at least one relevant label is not in common, between the application consumer genomic dataset and the reference genomic dataset, for a gene and associated outcome.

18. The computer-implemented method according to aspect 16, further comprising: generating, by the one or more processors, a cosine similarity between the application consumer genomic dataset and the reference genomic dataset.

19. The computer-implemented method according to any one of aspects 13 to 18, further comprising: verifying, by the one or more processors, the diagnostic prediction model of the one or more diagnostic prediction models for a second application consumer based upon receiving information corresponding to a second application consumer genomic dataset; authorizing, by the one or more processors, the diagnostic prediction model for distribution to the second application consumer; and providing, by the one or more processors, the diagnostic prediction model and the corresponding adaptation factors to the second application consumer.

20. A non-transitory, tangible computer-readable medium storing machine-readable instructions for distributing diagnostic prediction models that, when executed by one or more processors, cause the one or more processors to: receive from an application provider: one or more diagnostic prediction models, each model trained to generate a respective diagnostic prediction based upon genomic data; and for the each model, adaptation factors used to transform a target genomic dataset to conform to a dataset-specific nature of a reference genomic dataset of the each model; provide for display via a graphical user interface, one or more representations corresponding to the one or more diagnostic prediction models; verify a diagnostic prediction model of the one or more diagnostic prediction models for an application consumer based upon receiving information corresponding to an application consumer genomic dataset; authorize the diagnostic prediction model for distribution to the application consumer; and provide the diagnostic prediction model and the corresponding adaptation factors to the application consumer.

21. A computing system for improved trustless diagnostic prediction model sharing, comprising: an electronic network; one or more processors; and one or more memories having stored thereon computer-executable instructions that, when executed by the one or more processors, cause the computing system to: receive from an application provider: one or more diagnostic prediction applications, each including: a diagnostic prediction model which generates a diagnostic prediction based upon a genomic dataset; and a data transformer configured to transform a target genomic dataset to conform to a dataset-specific nature of a reference genomic dataset of the each model; and a dataset verifier which identifies at least one cohort of an application consumer genomic dataset for use by a diagnostic prediction application corresponding to the dataset verifier; receive from an application consumer, a request for a diagnostic prediction application of the one or more diagnostic prediction applications for a corresponding application consumer genomic dataset; in response to the request for the diagnostic prediction application, transmit to the application consumer, the dataset verifier corresponding to the diagnostic prediction application; receive from the application consumer, a verified request for the diagnostic prediction application indicating the dataset verifier has identified the at least one cohort of the application consumer genomic dataset for use by the diagnostic prediction application corresponding to the dataset verifier; and in response to the verified request, transmit to the application consumer, the diagnostic prediction application corresponding to the dataset verifier.

22. The computing system according to aspect 21, the one or more memories having stored thereon instructions that, when executed, cause the computing system further to: determine at least one relevant label in common, between the application consumer genomic dataset and the reference genomic dataset, for a gene and associated outcome.

23. The computing system according to aspect 22, wherein the gene and associated outcome is based upon risk genes and protective genes.

24. The computing system according to any one of aspects 21 to 23, the one or more memories having stored thereon instructions that, when executed, cause the computing system further to: determine pairs of genes which have at least one covariance between the application consumer genomic dataset and the reference genomic dataset.

25. The computing system according to aspect 24, wherein to determine of pairs of genes which have at least one covariance, the one or more memories having stored thereon instructions that, when executed, cause the computing system further to: determine at least one relevant label is not in common, between the application consumer genomic dataset and the reference genomic dataset, for a gene and associated outcome.

26. The computing system according to aspect 24, the one or more memories having stored thereon instructions that, when executed, cause the computing system to: generate a cosine similarity between the application consumer genomic dataset and the reference genomic dataset.

27. The computing system according to any one of aspects 21 to 26, the one or more memories having stored thereon instructions that, when executed, cause the computing system further to: receive from a second application consumer, a second request for the diagnostic prediction application; in response to the second request for the diagnostic prediction application, provide to the second application consumer, the dataset verifier; receive from the second application consumer, a second verified request for the diagnostic prediction application corresponding to the dataset verifier indicating the dataset verifier has identified at least one admissible cohort of a second application consumer genomic dataset for use by the diagnostic prediction application corresponding to the dataset verifier; and in response to the second verified request for the diagnostic prediction application corresponding to the dataset verifier, provide to the second application consumer, the diagnostic prediction application corresponding to the dataset verifier.

28. The computing system according to any one of aspects 21 to 27, wherein one or both of the application provider and the application consumer are affiliated with a biotechnology entity, an educational entity, a collaborator of a provider of the diagnostic prediction application, or a pharmaceutical entity.

29. The computing system according to any one of aspects 21 to 28, wherein the reference genomic dataset is generated by a first set of sequencing equipment and the application consumer genomic dataset is generated by a second set of sequencing equipment.

30. The computing system according to any one of aspects 21 to 29, wherein the reference genomic dataset and the application consumer genomic dataset have differences in characteristics due to their respective sets of sequencing equipment.

31. The computing system according to any one of aspects 21 to 30, wherein the reference genomic dataset is a public dataset or laboratory-specific dataset, and the application consumer genomic dataset is public dataset or laboratory-specific dataset.

32. The computing system according to any one of aspects 21 to 31, wherein the genomic prediction is a risk of cancer occurrence.

33. The computing system according to any one of aspects 21 to 32, wherein the diagnostic prediction application is at least one of a diagnosticator model, a prognosticator model, a subtyping model, or a predictive model.

34. The computing system according to aspect 33, the one or more memories having stored thereon instructions that, when executed, cause the computing system further to: process at least a first cohort of an application provider genomic dataset using the prognosticator model to generate an application provider gene association outcome; process at least a second cohort the application consumer genomic dataset using the prognosticator model to generate an application consumer gene association outcome; generate a statistical significance between the application provider gene association outcome and the application consumer gene association outcome; and determine, when the statistical significance is highly significant, that the prognosticator model is transferrable between the application provider and the application consumer.

35. The computing system according to aspect 33, the one or more memories having stored thereon instructions that, when executed, cause the computing system further to: receive a second genomic dataset, the reference genomic dataset, and one or more validation genomic datasets; generate a plurality of top genes correlated with the reference genomic dataset and the second genomic dataset; generate a result vector; for each validation genomic dataset of the one or more validation genomic datasets: generate a respective plurality of reference correlation values by processing the reference genomic dataset and the plurality of top genes; generate a respective plurality of validation correlation values by processing the each validation genomic dataset and the plurality of top genes; generate respective cosine similarity values between (i) each of the respective plurality of reference correlation values, and (ii) each of the respective plurality of validation correlation values; store the respective cosine similar values in the result vector; and cause the result vector to be transmitted to a remote device via the electronic network.

36. A method for automatically verifying a genomic dataset for use with a diagnostic prediction application, the method comprising: receiving one or more application consumer genomic datasets, and one or more application provider genomic datasets corresponding to a respective one or more diagnostic prediction models; determining, for each application provider genomic dataset of the one or more application provider genomic datasets, a gene-outcome correlation expressed by the each application provider genomic dataset; applying an algorithm to the one or more application consumer genomic datasets to determine, for each application consumer genomic dataset of the one or more application consumer genomic datasets, whether the gene-outcome correlation expressed by the each application provider genomic dataset is substantially similar to the gene-outcome correlation expressed by the one or more application consumer genomic datasets, wherein the gene-outcome correlation expressed by at least one of the one or more application consumer genomic datasets is substantially similar to the gene-outcome correlation expressed by at least one of the one or more application consumer genomic datasets, generating a gene-outcome validation dataset indicating the substantial similarities; and wherein the gene-outcome correlation expressed by at least one of the one or more application consumer genomic datasets is not substantially similar to the gene-outcome correlation expressed by at least one of the one or more application provider genomic datasets, apply the algorithm to the at least one of the one or more application consumer genomic datasets which are not substantially similar and the one or more application provider genomic datasets to determine pairs of genes which have at least one covariance between the at least one of the one or more application consumer genomic datasets which are not substantially similar and a respective application provider genomic dataset; and generate a gene-gene validation dataset indicating the covariance between the at least one of the one or more application consumer genomic datasets which are not substantially similar to a respective application provider genomic dataset.

37. The method according to aspect 36, wherein the gene-gene validation dataset indicates a cosine similarity between the at least one of the one or more application consumer genomic datasets which are not substantially similar and the respective application provider genomic dataset.

38. A computer-implemented method for rescuing failed drugs, the method comprising: receiving a diagnostic prediction model trained to prognosticate a genomic condition based upon a genomic dataset; analyzing, by the diagnostic prediction model, an historical genomic dataset indicative of a failed trial to generate a prognostication of the genomic condition; and outputting a report indicating the prognostication of the genomic condition.

39. A computer-implemented method for providing a plugin to an electronic health record system via a plug-in architecture of the electronic health record system, the computer-implemented method comprising: displaying, via a graphical user interface of an electronic health records client, a representation of one or more diagnostic prediction models, each trained to generate a respective diagnostic prediction based upon genomic data of an electronic health record; receiving, via the graphical user interface, a selection of at least one diagnostic prediction model of the one or more diagnostic prediction models; generating, at the electronic health records client, an electronic request including (i) an indication of the at least one diagnostic prediction model selected and (ii) one or more electronic health records of a patient including at least some genomic data; transmitting, via an electronic network, the electronic request to a remote computing device; receiving, from the remote computing device, an electronic result including one or more diagnostic predictions generated by the at least one diagnostic prediction models; and displaying, via the graphical user interface, the electronic result.

40. The computer-implemented method according to aspect 39, wherein the remote computing device includes: (i) one or more diagnostic prediction models, each model trained to generate a respective diagnostic prediction based upon genomic data; and (ii) one or more a data transformer configured to transform a target genomic dataset to conform to a dataset-specific nature a reference genomic dataset of the each model.

41. The computer-implemented method according to aspect 39 or 40, wherein the plug-in architecture is an Epic architecture or an FHIR architecture.

42. The computer-implemented method according to any one of aspects 39 to 41, wherein the diagnostic prediction includes a validation score indicating a validity of each diagnostic prediction based upon at least compatibility of the genomic data of the diagnostic prediction with the at least one diagnostic prediction model.

43. The computer-implemented method according to aspect 42, wherein the compatibility includes at least one relevant label in common, between the genomic data and reference genomic data of the at least one diagnostic prediction model, for a gene and associated outcome.

44. The computer-implemented method according to aspect 43, wherein the gene and associated outcome is based upon risk genes and protective genes.

45. The computer-implemented method according to aspect 42, wherein the compatibility includes pairs of genes which have at least one covariance between the genomic data and reference genomic data of the at least one diagnostic prediction model based upon determining at least one relevant label is not in common, between the genomic data and the reference genomic data of the at least one diagnostic prediction model, for a gene and associated outcome.

46. The computer-implemented method according to aspect 45, wherein the at least one diagnostic prediction model generates a cosine similarity between the genomic data and the reference genomic data.

47. The computer-implemented method according to aspect 40, wherein the reference genomic data is generated by a first set of sequencing equipment and the genomic data is generated by a second set of sequencing equipment.

48. The computer-implemented method according to aspect 47, wherein the reference genomic data and the genomic data have differences in characteristics due to their respective sets of sequencing equipment.

49. The computer-implemented method according to aspect 40, wherein the reference genomic data is a public dataset or laboratory-specific dataset, and the genomic data is public dataset or laboratory-specific dataset.

50. The computer-implemented method according to aspect 40, wherein the diagnostic prediction is a risk of cancer occurrence.

51. The computer-implemented method according to aspect 40, wherein the at least one diagnostic prediction model is at least one of a diagnosticator model, a prognosticator model, a subtyping model, or a predictive model.

52. The computer-implemented method according to aspect 51, wherein the at least one diagnostic prediction model, processes diagnostic prediction provider genomic data using the prognosticator model to generate a diagnostic prediction provider cohort response association outcome; processes the genomic data using the prognosticator model to generate a patient cohort response association outcome; generates a statistical significance between the diagnostic prediction provider cohort response association outcome and the patient cohort response association outcome; and determines, when the statistical significance is highly significant, that the prognosticator model is eligible for display via the graphical user interface of the electronic health records client.

### ADDITIONAL CONSIDERATIONS

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based upon the application of 35 U.S.C. § 112(f).

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in exemplary configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In exemplary embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special purpose processor, such as a field programmable gate array (FPGA) or an application-specific integrated circuit (ASIC) to perform certain operations). A hardware module may also comprise programmable logic or circuitry (e.g., as encompassed within a general purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general purpose processor configured using software, the general purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of exemplary methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some exemplary embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some embodiments, the processor or processors may be located in a single location (e.g., within a home environment, an office environment or as a server farm), while in other embodiments the processors may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, nonvolatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still cooperate or interact with each other. The embodiments are not limited in this context.

As used herein, the terms “comprises,” “comprising,” “includes,” “including,” “has,” “having” or any other variation thereof, are intended to cover a nonexclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Upon reading this disclosure, those of skill in the art will appreciate still additional alternative structural and functional designs for the approaches described herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

Furthermore, the patent claims at the end of this patent application are not intended to be construed under 35 U.S.C. 112 § (f) unless traditional means plus function language is expressly recited, such as “means for” or “step for” language being explicitly recited in the claim(s). The systems and methods described herein are directed to an improvement to computer functionality, and improve the functioning of conventional computers.

What is claimed is:

1. A computing system for distributing diagnostic prediction models comprising:
one or more memories having stored thereon computer-executable instructions that, when executed by one or more processors, cause the computing system to:
receive from an application provider:
one or more diagnostic prediction models, each model trained to generate a respective diagnostic prediction based upon genomic data; and
for the each model, adaptation factors used to transform a target genomic dataset to conform to a dataset-specific nature of a reference genomic dataset of the each model;
provide for display via a graphical user interface, one or more representations corresponding to the one or more diagnostic prediction models;
verify a diagnostic prediction model of the one or more diagnostic prediction models for an application consumer based upon receiving information corresponding to an application consumer genomic dataset;
authorize the diagnostic prediction model for distribution to the application consumer; and
provide the diagnostic prediction model and the corresponding adaptation factors to the application consumer.

2. The computing system of claim 1, the one or more memories having stored thereon instructions that, when executed, cause the computing system further to:
determine at least one relevant label in common, between the application consumer genomic dataset and the reference genomic dataset, for a gene and associated outcome.

3. The computing system of claim 2, wherein the gene and associated outcome is based upon risk genes and protective genes.

4. The computing system of claim 1, the one or more memories having stored thereon instructions that, when executed, cause the computing system further to:
determine pairs of genes which have at least one covariance between the application consumer genomic dataset and the reference genomic dataset.

5. The computing system of claim 4, wherein to determine pairs of genes which have at least one covariance, the one or more memories have stored thereon instructions that, when executed, cause the computing system further to:
determine at least one relevant label is not in common, between the application consumer genomic dataset and the reference genomic dataset, for a gene and associated outcome.

6. The computing system of claim 4, the one or more memories having stored thereon instructions that, when executed, cause the computing system to:
generate a cosine similarity between the application consumer genomic dataset and the reference genomic dataset.

7. The computing system of claim 1, the one or more memories having stored thereon instructions that, when executed, cause the computing system further to:
verify the diagnostic prediction model of the one or more diagnostic prediction models for a second application consumer based upon receiving information corresponding to a second application consumer genomic dataset;
authorize the diagnostic prediction model for distribution to the second application consumer; and
provide the diagnostic prediction model and the corresponding adaptation factors to the second application consumer.

8. The computing system of claim 1, wherein one or both of the application provider and the application consumer are affiliated with a biotechnology entity, an educational entity, a collaborator of a provider of the diagnostic prediction models, or a pharmaceutical entity.

9. The computing system of claim 1, wherein the reference genomic dataset is generated by a first set of sequencing equipment and the application consumer genomic dataset is generated by a second set of sequencing equipment.

10. The computing system of claim 9, wherein the reference genomic dataset and the application consumer genomic dataset have differences in characteristics due to their respective sets of sequencing equipment.

11. The computing system of claim 1, wherein the reference genomic dataset is a public dataset or a laboratory-specific dataset, and the application consumer genomic dataset is the public dataset or the laboratory-specific dataset.

12. The computing system of claim 1, wherein the diagnostic prediction model is at least one of a diagnosticator model, a prognosticator model, a subtyping model, or a predictive model.

13. A computer-implemented method for distributing diagnostic prediction models comprising:
receiving, via one or more processors, from an application provider:
one or more diagnostic prediction models, each model trained to generate a respective diagnostic prediction based upon genomic data; and
for the each model, adaptation factors used to transform a target genomic dataset to conform to a dataset-specific nature of a reference genomic dataset of the each model;
providing, by the one or more processors for display via a graphical user interface, one or more representations corresponding to the one or more diagnostic prediction models;
verifying, by the one or more processors, a diagnostic prediction model of the one or more diagnostic prediction models for an application consumer based upon receiving information corresponding to an application consumer genomic dataset;
authorizing, by the one or more processors, the diagnostic prediction model for distribution to the application consumer; and
providing, by the one or more processors, the diagnostic prediction model and the corresponding adaptation factors to the application consumer.

14. The computer-implemented method of claim 13, further comprising:
determining, by the one or more processors, at least one relevant label in common, between the application consumer genomic dataset and the reference genomic dataset, for a gene and associated outcome.

15. The computer-implemented method of claim 14, wherein the gene and associated outcome is based upon risk genes and protective genes.

16. The computer-implemented method of claim 13, further comprising:
determining, by the one or more processors, pairs of genes which have at least one covariance between the application consumer genomic dataset and the reference genomic dataset.

17. The computer-implemented method of claim 16, wherein determining the pairs of genes which have at least one covariance further comprises:
determining, by the one or more processors, at least one relevant label is not in common, between the application consumer genomic dataset and the reference genomic dataset, for a gene and associated outcome.

18. The computer-implemented method of claim 16, further comprising:
generating, by the one or more processors, a cosine similarity between the application consumer genomic dataset and the reference genomic dataset.

19. The computer-implemented method of claim 13, further comprising:
verifying, by the one or more processors, the diagnostic prediction model of the one or more diagnostic prediction models for a second application consumer based upon receiving information corresponding to a second application consumer genomic dataset;
authorizing, by the one or more processors, the diagnostic prediction model for distribution to the second application consumer; and
providing, by the one or more processors, the diagnostic prediction model and the corresponding adaptation factors to the second application consumer.

20. A non-transitory, tangible computer-readable medium storing machine-readable instructions for distributing diagnostic prediction models that, when executed by one or more processors, cause the one or more processors to:
receive from an application provider:
one or more diagnostic prediction models, each model trained to generate a respective diagnostic prediction based upon genomic data; and
for the each model, adaptation factors used to transform a target genomic dataset to conform to a dataset-specific nature of a reference genomic dataset of the each model;
provide for display via a graphical user interface, one or more representations corresponding to the one or more diagnostic prediction models;
verify a diagnostic prediction model of the one or more diagnostic prediction models for an application consumer based upon receiving information corresponding to an application consumer genomic dataset;
authorize the diagnostic prediction model for distribution to the application consumer; and
provide the diagnostic prediction model and the corresponding adaptation factors to the application consumer.
